(19) United States
(12) Patent Application Publication
(10) Pub. No.: US 2025/0204794 A1
(43) Pub. Date: Jun. 26, 2025

(54) COGNITIVE COMPUTING-BASED SYSTEM AND METHOD FOR NON-INVASIVE ANALYSIS OF PHYSIOLOGICAL INDICATORS USING ASSISTED TRANSDERMAL OPTICAL IMAGING

(71) Applicant: Aarogya Tech Inc., Reading, PA (US)

(72) Inventors: Amit Kumar Gupta, Bangalore (IN); Prashant Trivedi, Reading, PA (US); Prashant Gupta, Bangalore (IN)

(21) Appl. No.: 18/986,931

(22) Filed: Dec. 19, 2024

Related U.S. Application Data
(60) Provisional application No. 63/612,408, filed on Dec. 20, 2023.

(51) Int. Cl. A61B 5/0205; A61B 5/01; A61B 5/021; A61B 5/024; A61B 5/026; G06T 3/40; G06T 5/70; G06T 7/20; G06T 7/90; G06V 10/25; G06V 40/16

(52) U.S. Cl. CPC A61B 5/0205; A61B 5/01; A61B 5/021; A61B 5/02405; A61B 5/026; G06T 3/40; G06T 5/70; G06T 7/20; G06T 7/90; G06V 10/25; G06V 40/161; G06T 2207/10024; G06V 2201/07

(57) ABSTRACT

A cognitive computing-based system and a method for non-invasive analysis of one or more physiological indicators using assisted transdermal optical imaging are disclosed. The cognitive computing-based system is configured to obtain multi-modal data from a plurality of sources to preprocess the multi-modal data that includes colored image data, thermal image data, physiological data, and user-provided contextual data. The preprocessed data is used for generating one or more multi-modal features. The cognitive computing-based system is configured to generate unified features representation data for machine learning (ML) models analysis using the extracted one or more multi-modal features. Further, the cognitive computing-based system is configured to perform non-invasive analysis on the unified features representation data to detect temporal trends in the unified features representation data, and generate numerical predictive insights, visual representations data, categorical predictive insights, and recommendation data of the physiological indicators based on the non-invasive analysis of the physiological indicators.

### CROSS REFERENCE TO RELATED APPLICATION(S)

This application claims the priority to and incorporates by reference the entire disclosure of U.S. provisional patent application bearing No. 63/612,408 filed on Dec. 20, 2023; titled “A SYSTEM FOR ASSISTIVE TRANSDERMAL OPTICAL IMAGING AND METHOD THEREOF”.

### TECHNICAL FIELD

Embodiments of the present disclosure relate to transdermal optical imaging, and more particularly relate to a cognitive computing-based system and method for non-invasive analysis of one or more physiological indicators using assisted transdermal optical imaging.

### BACKGROUND

Physiological monitoring and analysis have experienced significant advancements due to integrating non-invasive technologies, multi-modal data collection, and machine learning models. However, existing systems face critical limitations in terms of accuracy, robustness, and adaptability when analyzing complex physiological indicators such as hemoglobin levels, stress, and heart rate variability.

Current physiological monitoring systems often rely on single data modalities, such as optical imaging, thermal imaging, or physiological sensors. While the physiological monitoring systems provide useful insights, they are prone to inaccuracies due to the inherent limitations of each modality of the single data modalities. Optical imaging systems are affected by motion artifacts, inconsistent lighting conditions, and variations in skin tone, leading to unreliable results. While useful for capturing temperature-related metrics, thermal imaging systems often lack contextual information needed to correlate physiological changes with health indicators. Physiological sensors, such as heart rate monitors and blood pressure cuffs, provide accurate single-point measurements but fail to capture comprehensive multi-dimensional insights about a health of users.

Existing technologies frequently lack an integrated approach for combining multi-modal data. This fragmentation results in a) Redundant or incomplete feature sets that fail to leverage cross-modal relationships, b) Increased dependency on modality-specific data, making the physiological monitoring systems prone to failure in case of noise or unavailability in one data source, and c) Limited ability to generate holistic insights or personalized health predictions.

Many of the physiological monitoring systems are configured for controlled environments (e.g., clinical or laboratory settings) and fail to perform consistently in dynamic, real-world conditions such as a) Variations in ambient lighting, temperature, and noise, b) Differences in user demographics, such as age, gender, and skin tone, and c) Changes in user behavior, such as facial movements, body posture, or lifestyle patterns.

While the machine learning models have been employed in health monitoring, many of the physiological monitoring systems use static models that lack adaptability. Challenges include: the machine learning models trained on static datasets often fail to adapt to new data patterns or changing user conditions. Most physiological monitoring systems lack mechanisms for learning from user feedback or clinician inputs to improve predictions over time. The physiological monitoring systems are capable of simultaneously predicting multiple physiological and mental health indicators, leading to inefficiencies in analysis and resource utilization.

Existing physiological monitoring systems often focus on retrospective analysis rather than real-time predictions, limiting their ability to provide actionable insights in time-sensitive scenarios. Additionally, outputs from many machine learning models lack transparency, making it difficult for the users to trust or interpret the results effectively. Further, sophisticated health monitoring technologies are often costly, require specialized hardware, and are difficult to use in home or non-clinical settings. These limitations restrict widespread adoption and accessibility, especially in resource-constrained environments. Furthermore, the current physiological monitoring systems often ignore contextual data, such as user lifestyle, sleep patterns, and emotional states, which may play a significant role in understanding physiological health indicators. Furthermore, temporal trends in physiological data, such as circadian patterns and long-term health changes, are frequently overlooked, leading to incomplete assessments.

Therefore, there is a need for a cognitive computing-based system to address the aforementioned issues by providing a solution for non-invasive analysis of one or more physiological indicators using assisted transdermal optical imaging.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a cognitive computing-based method for non-invasive analysis of one or more physiological indicators using assisted transdermal optical imaging is disclosed. In a first step, the cognitive computing-based method includes obtaining, by one or more servers through a data-obtaining subsystem, multi-modal data from a plurality of sources comprising at least one of: one or more image-capturing units, one or more thermal imaging sensors, one or more physiological sensing peripherals, and one or more users. The multi-modal data comprises at least one of: colored image data, thermal image data, physiological data, speech signals, and user-provided contextual data. The one or more image-capturing units comprise at least one of: a high-resolution optical camera, a near-infrared (NIR) camera, a hyperspectral imaging sensor, a multispectral camera, a time-of-flight (ToF) camera, a photoplethysmography (PPG) imaging sensor, a polarized light camera, and a micro-imaging sensor. The one or more thermal imaging sensors comprise at least one of: an infrared thermal imaging sensor, a long-wave infrared (LWIR) sensor, a mid-wave infrared (MWIR) sensor, a short-wave infrared (SWIR) sensor, a far-infrared (FIR) sensor, a microbolometer-based thermal sensor, a thermopile array sensor, and a thermal imaging sensor integrated with visible-light imaging.

The one or more physiological sensing peripherals comprise at least one of: heart rate monitors, blood pressure monitors, respiratory rate sensors, pulse oximeters, body weighing scales, an electrocardiogram (ECG), Non-Contact IR Thermometer (NCIT), and body impedance analyzers. The colored image data comprises at least one of: pixel-level blood flow patterns, hemoglobin concentration, oxygen saturation, and the micro-expression detection data in defined facial regions, including at least one of: cheeks, a forehead, nose, nasal cavity, and inner eyelids. The thermal image data comprises at least one of: temperature distribution patterns, inflammation indicators, deeper tissue activity data, and blood flow dynamics across facial regions of the one or more users. The physiological data comprises at least one of: heart rate data, blood pressure data, respiratory rate data, oxygen level data, body weight data, body temperature, and body impedance data. The user-provided contextual data comprises at least one of: user-reported stress levels; sleep duration; lifestyle factors, including at least one of: physical activity level and dietary habits; medication history; self-reported symptoms, including at least one of: fatigue, dizziness, and pain; personal medical history, including at least one of: pre-existing conditions and diagnoses; and demographic information including at least one of: age, gender, and occupation.

In an embodiment, the cognitive computing-based method includes obtaining the speech signals using one or more speech processors to extract one or more acoustic features indicative of at least one of: emotional variability, fatigue, and stress levels. The one or more acoustic features comprise at least one of: a pitch, a tone, a speech rhythm, and a voice intensity, processed by one or more machine learning (ML) models to determine the one or more physiological indicators.

In next step, the cognitive computing-based method includes preprocessing, by the one or more servers through a data preprocessing subsystem, the obtained multi-modal data comprises: a) preprocessing the colored image data based on performing at least one of: a face detection and alignment, a motion compensation, a standardization, an artifact removal, and a region-of-interest (ROI) isolation, b) preprocessing the thermal image data based on performing at least one of: a temperature calibration, a noise reduction, and a region-of-interest (ROI) tracking to capture at least one of: temperature patterns, blood flow, and inflammation markers, c) preprocessing the physiological data based on performing at least one of: a signal filtering, a baseline correction, a feature extraction for raw signals, a data synchronization with at least one of: timestamps and one or more physiological event markers, outlier detection and exclusion, time normalization, unit standardization, and segmentation of the physiological data at pre-defined intervals; and d) preprocessing the user-provided contextual data to perform at least one of: data encoding, authorizing contextual data, and computing composite scores.

In yet another embodiment, the cognitive computing-based method includes preprocessing the colored image data comprise at least one of: a) determining the facial regions of the one or more users using one or more object detection models to ensure the region-of-interest (ROI) is consistent across one or more image frames in the colored image data, b) performing at least one of: the motion compensation process and the region-of-interest (ROI) tracking on the colored image data for stabilizing the region-of-interest (ROI) across the one or more image frames, c) normalizing at least one of: brightness, contrast, and color of the colored image data associated to account for differences in lighting conditions, and d) scaling one or more images in the colored image data to a defined dimension for providing uniform input for the one or more machine learning (ML) models.

In yet another embodiment, the cognitive computing-based method includes preprocessing the thermal image data further comprises aligning the thermal image data with the corresponding-colored image data by mapping the regions of interest (ROI) to ensure spatial and temporal consistency across the multi-modal data. In yet another embodiment, the cognitive computing-based method includes processing the physiological data using at least one of: a Z-score normalization model, a min-max scaling model, a robust scaling model for at least one of: normalizing the physiological data, standardizing the physiological data, and perform the outlier detection and exclusion. In yet another embodiment, the cognitive computing-based method includes preprocessing the user-provided contextual data further comprises a) processing the user-provided contextual data using at least one of: a one-hot encoding model, an ordinal encoding model, and a binary encoding model, as the one or more machine learning (ML) models for at least one of: converting into categorical responses, converting into scaled responses, and processing one of: yes and no contextual data, and b) extracting at least one of: symptom severity scores, risk factor aggregation, temporal change in the contextual data, combined health indicators, and mental state indicators of the one or more users based on the processed user-provided contextual data.

In the next step, the cognitive computing-based method includes generating, by the one or more servers through a feature engineering subsystem, one or more multi-modal features based on: a) extracting at least one of: blood flow pattern data, hemoglobin estimation data, tissue perfusion characteristics, and micro-expression detection data from the colored image data based on analyzing at least one of: pixel-level changes, a color spectrum, and subtle facial movements, in the preprocessed colored image data, b) extracting at least one of: temperature distribution patterns, inflammation indicator data, and blood flow dynamics from the thermal image data using at least one of: one or more machine learning (ML) models and image filtering models, c) generating at least one of: time-domain heart rate variability (HRV) features, frequency-domain heart rate variability (HRV) features, systolic and diastolic blood pressure ratios, respiratory variability, stress and cardiovascular markers, and oxygen saturation fluctuation data from the physiological data using the one or more machine learning (ML) models, and d) determining at least one of: encoded features and derived metrics from the user-provided contextual data.

In yet another embodiment, the cognitive computing-based method includes generating the one or more multi-modal features using one or more convolutional neural networks (CNNs) as the one or more machine learning (ML) models. The cognitive computing-based method includes generating at least one of: the time-domain heart rate variability (HRV) features and the frequency-domain heart rate variability (HRV) features, including a first-frequency, a second-frequency, and the first-frequency and the second-frequency ratios, to evaluate autonomic nervous system activity and stress and cardiovascular markers.

In the next step, the cognitive computing-based method includes generating, by the one or more servers through a feature fusion subsystem, unified features representation data comprises: a) synchronizing the multi-modal data from the plurality of sources using at least one of: the timestamps and the one or more physiological event markers as one or more anchor points, b) integrating the generated one or more multi-modal features into the unified features representation data for the one or more machine learning (ML) models analysis, and c) assigning one or more domain-specific constraints to the unified features representation data to alleviate one or more physiologically inapt combinations.

In yet another embodiment, the cognitive computing-based method includes generating the unified features representation data comprises: using at least one of: one or more modality-specific attention procedures, a cross-modal relationship learning model, one or more adaptive feature importance weighting procedures, to: a) integrating the generated one or more multi-modal features into unified features representation data, and b) assigning one or more weights to each multi-modal feature of the one or more multi-modal features based on reliability and relevance of the one or more multi-modal features for predicting the one or more physiological indicators.

In the next step, the cognitive computing-based method includes analyzing, by the one or more servers through a data analysis subsystem, the unified features representation data using the one or more machine learning (ML) models comprises: a) performing non-invasive analysis on the unified features representation data based on generating a data correlation with one or more labeled datasets to predict the one or more physiological indicators, b) detecting one or more temporal trends in the unified features representation data including at least one of: change in the one or more physiological indicators over time, recurring patterns in the physiological data, and one or more abnormal health conditions, and c) generating at least one of: one or more numerical predictive insights, visual representations data, one or more categorical predictive insights, and recommendation data of the one or more physiological indicators based on the non-invasive analysis of the one or more physiological indicators. The one or more physiological indicators comprise at least one of: hemoglobin concentration, glycated hemoglobin (HbA1c), blood pressure, blood glucose, heart rate variability (HRV), blood oxygen saturation (SpO2), respiratory rate, stress levels, cognitive load, fatigue, and emotional variability. The one or more machine learning (ML) models comprise at least one of: an XGBoost Regressor, the one or more convolutional neural networks (CNNs) with a long short-term memory (LSTM) hybrid, a random forest, a bidirectional long short-term memory (BiLSTM), a transformer-convolutional neural network (CNN), recurrent neural networks (RNNs), light gradient-boosting machine (LightGBM), a transformer-based multi-task model, deep neural network, gradient boosting, neural networks, a k-means clustering, a hierarchical clustering and support vector machines (SVMs).

In yet another embodiment, the data analysis subsystem is configured to select the one or more machine learning (ML) models based on a type of physiological indicators being predicted within the one or more physiological indicators, wherein the one or more machine learning (ML) models include at least one of: a) one or more supervised learning models for labeled data within the multi-modal data, including: one or more regression models for determining at least one of: the hemoglobin concentration and the glycated hemoglobin (HbA1c), using at least one of: the XGBoost Regressor and the random forest, and one or more classification models for determining categorical outputs, comprise at least one of: the stress levels and inflammation markers, using at least one of: the support vector machines (SVMs) and the one or more convolutional neural networks (CNNs), b) one or more unsupervised learning models for unlabeled data within the multi-modal data, including: one or more clustering models comprises at least one of: the k-means clustering and the hierarchical clustering, for identifying one or more multi-modal feature patterns within the unified features representation data of the one or more users based on the one or more multi-modal features extracted from the multi-modal data, and c) one or more deep learning models including at least one of: the recurrent neural networks (RNNs), the transformer-convolutional neural network (CNN), and the long short-term memory (LSTM) hybrid, for analyzing at least one of: the blood flow patterns, the hemoglobin estimation data, the tissue perfusion characteristics, and the micro-expression detection data in the colored image data.

In yet another embodiment, the cognitive computing-based method includes training the one or more machine learning (ML) models using at least one of: one or more loss function models, stochastic gradient descent (SGD), Adaptive Moment Estimation (Adam), Root Mean Square Propagation (RMSprop), Hyperparameter tuning models, for at least one of: continuous variable prediction, classification of the multi-modal data, and alleviate the one or more physiologically inapt combinations. In yet another embodiment, the cognitive computing-based method includes adaptively retraining the one or more machine learning (ML) models on real-time acquired multi-modal data to optimize prediction accuracy and address multi-modal data drift caused by variations in at least one of: the demographic information, environmental conditions, and one or more sensor settings. In yet another embodiment, the cognitive computing-based method includes incorporating one or more feedback loops from the one or more users to refine the predictions and outputs of the one or more machine learning (ML) models over time.

In accordance with another embodiment of the present disclosure, a cognitive computing-based system for non-invasive analysis of the one or more physiological indicators using the assisted transdermal optical imaging. The cognitive computing-based system comprises the one or more servers configured with one or more hardware processors and a memory unit. The memory unit is operatively connected to the one or more hardware processors. The memory unit comprises a set of computer-readable instructions in form of a plurality of subsystems, configured to be executed by the one or more hardware processors. The plurality of subsystems comprises the data-obtaining subsystem, the data preprocessing subsystem, the feature engineering subsystem, the feature fusion subsystem, and the data analysis subsystem.

In an embodiment, the data-obtaining subsystem is configured to obtain the multi-modal data from the plurality of sources comprise at least one of: the one or more image-capturing units, the one or more thermal imaging sensors, the one or more physiological sensing peripherals, and the one or more users. The multi-modal data comprises at least one of: the colored image data, the thermal image data, the physiological data, the speech signals, and the user-provided contextual data.

Yet another embodiment, the data preprocessing subsystem configured to: a) preprocess the colored image data based on performing at least one of: the face detection and alignment, the motion compensation, the standardization, the artifact removal, and the region-of-interest (ROI) isolation, b) preprocess the thermal image data based on performing at least one of: the temperature calibration, the noise reduction, the region-of-interest (ROI) tracking to capture at least one of: temperature patterns, blood flow, and inflammation markers, c) preprocess the physiological data based on performing at least one of: the signal filtering, the baseline correction, the feature extraction for raw signals, the data synchronization with at least one of: the timestamps and the one or more physiological event markers, the outlier detection and exclusion, the time normalization, the unit standardization, and the segmentation of the physiological data at the pre-defined intervals, and d) preprocess the user-provided contextual data to perform at least one of: the data encoding, the authorizing contextual data, and the computing composite scores.

Yet another embodiment, the feature engineering subsystem configured to generate one or more multi-modal features based on: a) extracting at least one of: the blood flow pattern data, the hemoglobin estimation data, the tissue perfusion characteristics, and the micro-expression detection data from the colored image data based on analyzing at least one of: the pixel-level changes, the color spectrum, and the subtle facial movements, in the preprocessed colored image data, b) extracting at least one of: the temperature distribution patterns, the inflammation indicator data, and the blood flow dynamics from the thermal image data using at least one of: the one or more machine learning (ML) models and the one or more image filtering models, c) generating at least one of: the time-domain heart rate variability (HRV) features, the frequency-domain heart rate variability (HRV) features, the systolic and diastolic blood pressure ratios, the respiratory variability, the stress and cardiovascular markers, and the oxygen saturation fluctuation data from the physiological data using the one or more machine learning (ML) models, and d) determining at least one of: encoded features and derived metrics from the user-provided contextual data.

Yet another embodiment, the feature fusion subsystem is configured to: a) synchronize the multi-modal data from the plurality of sources using at least one of: the timestamps and the one or more physiological event markers as the one or more anchor points, b) integrate the generated the one or more multi-modal features into unified features representation data for the one or more machine learning (ML) models analysis, and c) assigning one or more domain-specific constraints to the unified features representation data to alleviate one or more physiologically inapt combinations.

Yet another embodiment, the data analysis subsystem configured with the one or more machine learning (ML) models to: a) perform non-invasive analysis on the unified features representation data based on generating the data correlation with the one or more labeled datasets to predict the one or more physiological indicators, b) detect the one or more temporal trends in the unified features representation data including at least one of: change in the one or more physiological indicators over time, recurring patterns in the physiological data, and one or more abnormal health conditions, and c) generate at least one of: the one or more numerical predictive insights, the visual representations data, the one or more categorical predictive insights, and the recommendation data of the one or more physiological indicators based on the non-invasive analysis of the one or more physiological indicators.

In accordance with another exemplary embodiment of the present disclosure, a non-transitory computer-readable storage medium storing computer-executable instructions that, when executed by the one or more hardware processors, cause the one or more hardware processors to perform operations for non-invasive analysis of one or more physiological indicators using assisted transdermal optical imaging. The operations comprising: a) obtaining the multi-modal data from the plurality of sources comprise at least one of: the one or more image-capturing units, the one or more thermal imaging sensors, the one or more physiological sensing peripherals, and the one or more users, b) preprocessing the obtained multi-modal data comprises: i) preprocessing the colored image data based on performing at least one of: the face detection and alignment, the motion compensation, the standardization, the artifact removal, and the region-of-interest (ROI) isolation, ii) preprocessing the thermal image data based on performing at least one of: the temperature calibration, the noise reduction, and the region-of-interest (ROI) tracking to capture at least one of: the temperature patterns, the blood flow, and the inflammation markers, iii) preprocessing the physiological data based on performing at least one of: the signal filtering, the baseline correction, the feature extraction for raw signals, the data synchronization with at least one of: the timestamps and the one or more physiological event markers, the outlier detection and exclusion, the time normalization, the unit standardization, and the segmentation of the physiological data at pre-defined intervals, and iv) preprocessing the user-provided contextual data to perform at least one of: the data encoding, the authorizing contextual data, and the computing composite scores, c) generating one or more multi-modal features based on: i) extracting at least one of: the blood flow pattern data, the hemoglobin estimation data, the tissue perfusion characteristics, and the micro-expression detection data from the colored image data based on analyzing at least one of: the pixel-level changes, the color spectrum, and the subtle facial movements, in the preprocessed colored image data, ii) extracting at least one of: the temperature distribution patterns, the inflammation indicator data, and the blood flow dynamics from the thermal image data using at least one of: the one or more machine learning (ML) models and image filtering models, iii) generating at least one of: the time-domain heart rate variability (HRV) features, the frequency-domain heart rate variability (HRV) features, the systolic and diastolic blood pressure ratios, the respiratory variability, the stress and cardiovascular markers, and the oxygen saturation fluctuation data from the physiological data using one or more machine learning (ML) models, and iv) determining at least one of: the encoded features and the derived metrics from the user-provided contextual data, d) generating the unified features representation data comprises: i) synchronizing the multi-modal data from the plurality of sources using at least one of: the timestamps and the one or more physiological event markers as one or more anchor points, ii) integrating the generated one or more multi-modal features into the unified features representation data for the one or more machine learning (ML) models analysis, and iii) assigning the one or more domain-specific constraints to the unified features representation data to alleviate the one or more physiologically inapt combinations, and e) analyzing the unified features representation data using the one or more machine learning (ML) models comprises: i) performing non-invasive analysis on the unified features representation data based on generating the data correlation with the one or more labeled datasets to predict the one or more physiological indicators, ii) detecting the one or more temporal trends in the unified features representation data including at least one of: change in the one or more physiological indicators over time, recurring patterns in the physiological data, and one or more abnormal health conditions, and iii) generating at least one of: the one or more numerical predictive insights, the visual representations data, the one or more categorical predictive insights, and the recommendation data of the one or more physiological indicators based on the non-invasive analysis of the one or more physiological indicators.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIGS. 1A-1C illustrate exemplary block diagram representations of a network architecture implementing a cognitive computing-based system for non-invasive analysis of one or more physiological indicators using assisted transdermal optical imaging, in accordance with an embodiment of the present disclosure;

FIG. 2A illustrates an exemplary block diagram representation of the cognitive computing-based system as shown in FIG. 1A, for non-invasive analysis of one or more physiological indicators using assisted transdermal optical imaging, in accordance with an embodiment of the present disclosure;

FIG. 2B illustrates an exemplary flow diagram representation of the cognitive computing-based system as shown in FIG. 2A, for non-invasive analysis of one or more physiological indicators using assisted transdermal optical imaging, in accordance with an embodiment of the present disclosure;

FIG. 3 illustrates an exemplary mental-marker flow architecture, in accordance with an embodiment of the present disclosure;

FIG. 4 illustrates an exemplary bio-marker flow architecture, in accordance with an embodiment of the present disclosure;

FIG. 5 illustrates an exemplary machine learning specific flow architecture, in accordance with an embodiment of the present disclosure

FIG. 6 illustrates an exemplary flow chart of a cognitive computing-based method for non-invasive analysis of the one or more physiological indicators using the assisted transdermal optical imaging, in accordance with an embodiment of the present disclosure; and

FIG. 7 illustrates an exemplary block diagram representation of one or more server platforms for implementation of the disclosed cognitive computing-based system, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module includes dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired), or temporarily configured (programmed) to operate in a certain manner and/or to perform certain operations described herein.

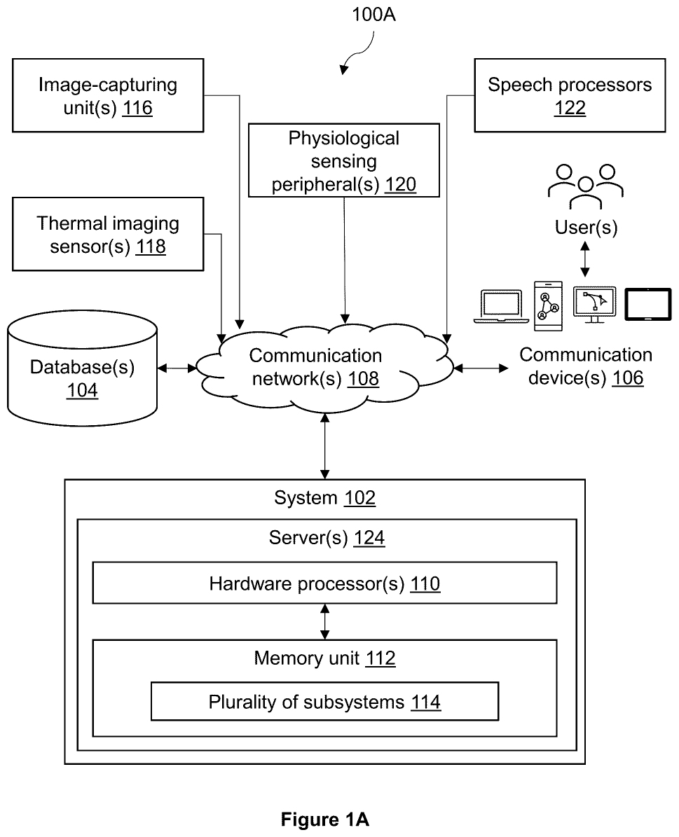

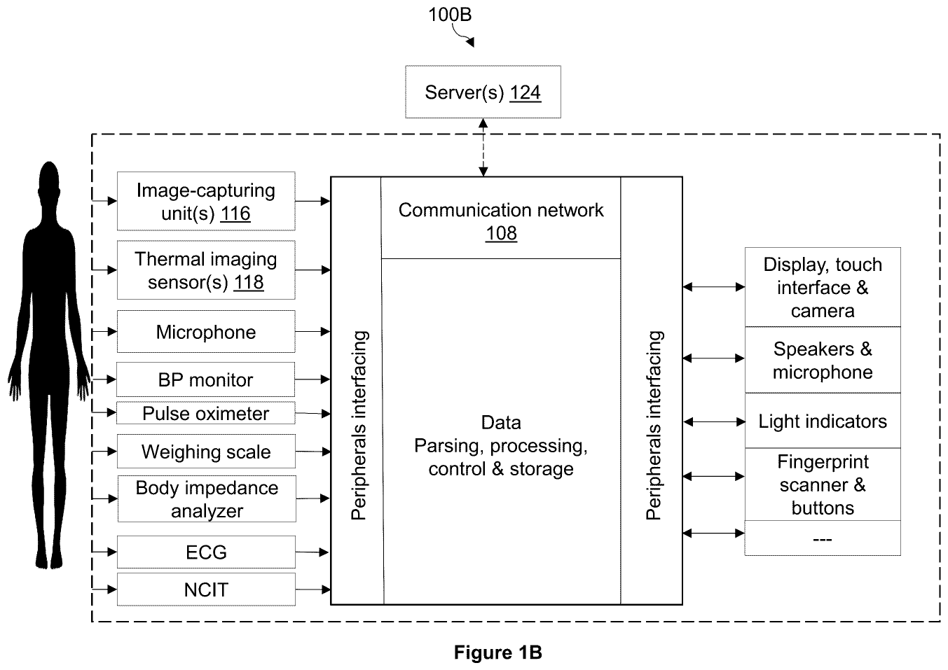

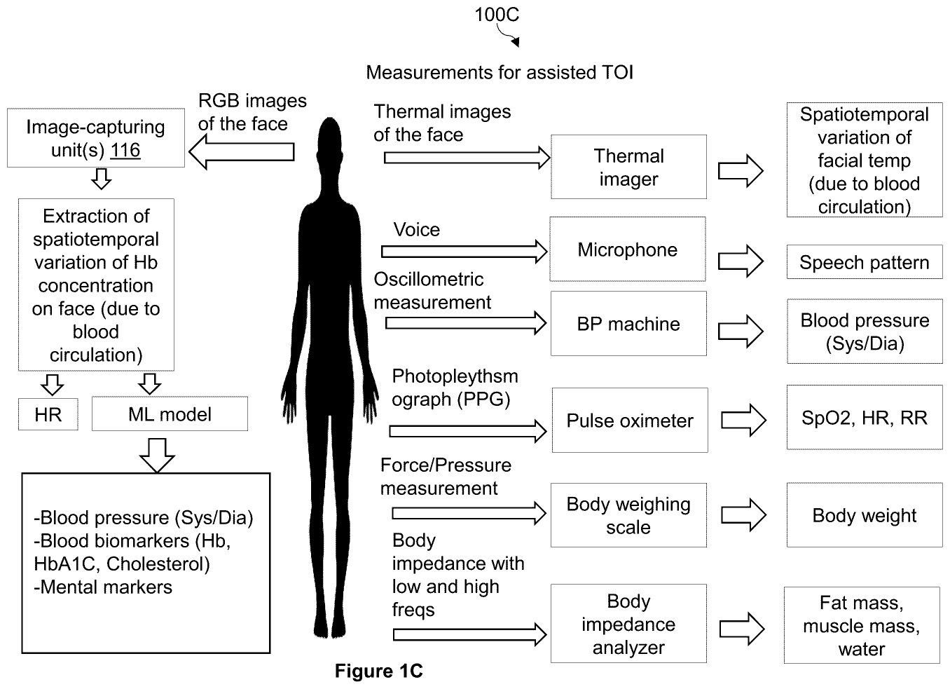

Referring now to the drawings, and more particularly to FIG. 1A through FIG. 7, where similar reference characters denote corresponding features consistently throughout the figures, there are shown preferred embodiments, and these embodiments are described in the context of the following exemplary system and/or method.

FIGS. 1A-1C illustrate exemplary block diagram representations of a network architecture (100A, 100B, 100C) implementing a cognitive computing-based system 102 for non-invasive analysis of one or more physiological indicators using assisted transdermal optical imaging, in accordance with an embodiment of the present disclosure.

According to an exemplary embodiment of the present disclosure, the network architecture (100A, 100B, 100C) may include the cognitive computing-based system 102, one or more databases 104, one or more communication devices 106, one or more image-capturing units 116, one or more thermal imaging sensors 118, one or more physiological sensing peripherals 120, and one or more speech processors 122. The cognitive computing-based system 102, the one or more databases 104, the one or more communication devices 106, the one or more image-capturing units 116, the one or more thermal imaging sensors 118, the one or more physiological sensing peripherals 120, and the one or more speech processors 122 may be communicatively coupled via one or more communication networks 108, ensuring seamless data transmission, processing, and the non-invasive analysis of the one or more physiological indicators. The cognitive computing-based system 102 acts as the central processing unit within the network architecture (100A, 100B, 100C), responsible for non-invasive analysis of the one or more physiological indicators using the assisted transdermal optical imaging.

In an exemplary embodiment, the cognitive computing-based system 102 may be deployed via one or more servers 124. The one or more servers 124 comprise one or more hardware processors 110 and a memory unit 112 that includes a set of computer-readable instructions executable by the one or more hardware processors 110 to analysis of the one or more physiological indicators using the assisted transdermal optical imaging.

The one or more hardware processors 110 may comprise a combination of discrete components, an integrated circuit, an application-specific integrated circuit, a field-programmable gate array, a digital signal processor, or other suitable one or more hardware processors 110 and a software. The “software” may comprise one or more objects, agents, threads, lines of code, subroutines, separate software applications, two or more lines of code, or other suitable software structures operating in one or more software applications or the one or more hardware processors 110. The memory unit 112 is operatively connected to the one or more hardware processors 110. The memory unit 112 comprises the set of computer-readable instructions in form of a plurality of subsystems 114, configured to be executed by the one or more hardware processors 110.

In an exemplary embodiment, the one or more hardware processors 110 may include, for example, microprocessors, microcomputers, microcontrollers, digital signal processors, central processing units, state machines, logic circuits, and/or any devices that manipulate data or signals based on operational instructions. Among other capabilities, the one or more hardware processors 110 may fetch and execute computer-readable instructions in the memory unit 112 operationally coupled with the cognitive computing-based system 102 for analyzing of one or more physiological indicators. Any reference to a task in the present disclosure may refer to an operation being or that may be performed on data. The one or more hardware processors 110 is high-performance processors capable of handling large volumes of data and complex computations. The one or more hardware processors 110 may be, but not limited to, at least one of: multi-core central processing units (CPU), graphics processing units (GPUs), and specialized Artificial Intelligence (AI) accelerators that enhance an ability of the cognitive computing-based system 102 to process real-time data from a plurality of sources simultaneously.

In an exemplary embodiment, the one or more databases 104 may configured to store, and manage data related to various aspects of the cognitive computing-based system 102. The one or more databases 104 may store at least one of, but not limited to, multi-modal data, preprocessed data, one or more multi-modal features, unified features representation data, one or more labeled datasets, machine learning models, historical data, feedback data, prediction outputs, system logs and diagnostic data, and the like. The one or more databases 104 may be regularly updated and synchronized to ensure consistency and availability of critical information. Additionally, the one or more databases 104 may be configured to provide rapid access to stored data for real-time processing, enabling the cognitive computing-based system 102 to dynamically adjust the one or more security policies and traffic management strategies based on the most current data available. The one or more databases 104 enable the cognitive computing-based system 102 to dynamically retrieve, analyze, and update the stored data in real-time, facilitating continuous analysis of the one or more physiological indicators using the assisted transdermal optical imaging. The one or more databases 104 may include different types of databases such as, but not limited to, relational databases (e.g., Structured Query Language (SQL) databases), non-Structured Query Language (NoSQL) databases (e.g., MongoDB, Cassandra), time-series databases (e.g., InfluxDB), an OpenSearch database, object storage systems (e.g., Amazon S3, PostgresDB), and the like.

In an exemplary embodiment, the one or more communication devices 106 are configured to enable one or more users to interact with the cognitive computing-based system 102. The one or more communication devices 106 may be digital devices, computing devices, and/or networks. The one or more communication devices 106 may include, but not limited to, a mobile device, a smartphone, a personal digital assistant (PDA), a tablet computer, a phablet computer, a wearable computing device, a virtual reality/augmented reality (VR/AR) device, a laptop, a desktop, and the like. The one or more communication devices 106 are configured with a user interface configured to enable seamless interaction between the one or more users and the cognitive computing-based system 102. The user interface may include the graphical user interfaces (GUIs), voice-based interfaces, and touch-based interfaces, depending on the capabilities of the one or more communication devices 106 being used. The GUIs may be designed to display outputs, including at least one of: one or more numerical predictive insights, visual representations data such as dashboards, graphs, and heatmaps, in an intuitive and user-friendly manner, one or more categorical predictive insights, and recommendation data of the one or more physiological indicators.

The user interface may also include interactive elements such as forms, drop-down menus, sliders, and buttons, allowing the one or more users to input data, provide feedback, and modify settings. For instance, the one or more users may submit user-provided contextual data, such as, but not limited to, at least one of: user-reported stress levels; sleep duration; lifestyle factors, including at least one of: physical activity level and dietary habits; medication history; self-reported symptoms, including at least one of: fatigue, dizziness, and pain; personal medical history, including at least one of: pre-existing conditions and diagnoses; and demographic information including at least one of: age, gender, and occupation, through input forms, or review and confirm system-generated predictions through interactive dialogs. The voice-based interfaces may enable the one or more users to provide speech signals to the cognitive computing-based system 102, receive verbal feedback, or issue commands through natural language processing (NLP) capabilities.

Additionally, touch-based interfaces may allow the one or more users to navigate the cognitive computing-based system 102 through gestures or touch commands, particularly on the mobile devices and the tablets. The user interface are further configured to support accessibility features, such as text-to-speech, high-contrast modes, and large text sizes, to ensure usability for the one or more users with varying abilities. Furthermore, the user interfaces may provide at least one of: one or more notifications and one or more alerts, such as health warnings or reminders, in real-time, ensuring that the one or more users remain informed and may act promptly. By offering versatile, the user interfaces, the one or more communication devices 106 ensure an efficient, accessible, and engaging user experience with the cognitive computing-based system 102.

In an exemplary embodiment, the one or more communication devices 106 may be associated with, but not limited to, one or more service providers, one or more customers, an individual, an administrator, a vendor, a technician, a specialist, an instructor, a supervisor, a team, an entity, an organization, a company, a facility, a bot, any other user, and combination thereof. The entity, the organization, and the facility may include, but not limited to, an e-commerce company, online marketplaces, service providers, retail stores, a merchant organization, a logistics company, warehouses, transportation company, an airline company, a hotel booking company, a hospital, a healthcare facility, an exercise facility, a laboratory facility, a company, an outlet, a manufacturing unit, an enterprise, an organization, an educational institution, a secured facility, a warehouse facility, a supply chain facility, any other facility/organization and the like.

In an exemplary embodiment, the one or more communication networks 108 may be, but not limited to, a wired communication network and/or a wireless communication network, a local area network (LAN), a wide area network (WAN), a Wireless Local Area Network (WLAN), a metropolitan area network (MAN), a telephone network, such as the Public Switched Telephone Network (PSTN) or a cellular network, an intranet, the Internet, a fiber optic network, a satellite network, a cloud computing network, or a combination of networks. The wired communication network may comprise, but not limited to, at least one of: Ethernet connections, Fiber Optics, Power Line Communications (PLCs), Serial Communications, Coaxial Cables, Quantum Communication, Advanced Fiber Optics, Hybrid Networks, and the like. The wireless communication network may comprise, but not limited to, at least one of: wireless fidelity (wi-fi), cellular networks (including fourth generation (4G) technologies and fifth generation (5G) technologies), Bluetooth, ZigBee, long-range wide area network (LoRaWAN), satellite communication, radio frequency identification (RFID), 6G (sixth generation) networks, advanced IoT protocols, mesh networks, non-terrestrial networks (NTNs), near field communication (NFC), and the like.

In an exemplary embodiment, the one or more image-capturing units 116 are configured to capture colored image data. The one or more image-capturing units 116 are configured to extract spatiotemporal variation of hemoglobin concentration of the face, caused due to blood circulation from the captured colored image data. The one or more image-capturing units 116 comprise, but not limited to, at least one of: a high-resolution optical camera, a near-infrared (NIR) camera, a hyperspectral imaging sensor, a multispectral camera, a time-of-flight (ToF) camera, a photoplethysmography (PPG) imaging sensor, a polarized light camera, a micro-imaging sensor, and the like. The colored image data comprises, but not limited to, at least one of: RGB images, pixel-level blood flow patterns, hemoglobin concentration, oxygen saturation, and the micro-expression detection data in defined facial regions, including at least one of: cheeks, a forehead, nose, nasal cavity, inner eyelids, and the like. The one or more image-capturing units 116 are further configured to operate under diverse ambient conditions, including variable lighting environments, and may incorporate features such as automatic exposure adjustment and noise reduction to enhance image quality. The one or more image-capturing units 116 are configured to integrate seamlessly with the cognitive computing-based system 102, ensuring that the captured colored image data is preprocessed and analyzed efficiently for non-invasive determination of the one or more physiological indicators.

In an exemplary embodiment, the one or more thermal imaging sensors 118 are configured to capture thermal image data facilitate spatiotemporal variation of facial temperature. The one or more thermal imaging sensors 118 comprise, but not limited to, at least one of: an infrared thermal imaging sensor, a long-wave infrared (LWIR) sensor, a mid-wave infrared (MWIR) sensor, a short-wave infrared (SWIR) sensor, a far-infrared (FIR) sensor, a microbolometer-based thermal sensor, a thermopile array sensor, a thermal imaging sensor integrated with visible-light imaging, and the like. The thermal image data comprises at least one of: temperature distribution patterns, inflammation indicators, deeper tissue activity data, and blood flow dynamics across facial regions of the one or more users.

In an exemplary embodiment, the one or more physiological sensing peripherals 120 are configured to determine physiological data. The one or more physiological sensing peripherals 120 comprise, but not limited to, at least one of: heart rate monitors, blood pressure monitors, electrocardiogram (ECG), Non-Contact IR Thermometer (NCIT), respiratory rate sensors, pulse oximeters, body weighing scales, body impedance analyzers, and the like. The physiological data comprises, but not limited to, at least one of: heart rate data, blood pressure data, respiratory rate data, oxygen level data, body weight data, body temperature, body impedance data, and the like.

In an exemplary embodiment, the one or more speech processors 122 are configured to are configured to analyze speech signals obtained from the one or more users to extract features indicative of physiological and emotional states. The one or more speech processors 122 may include hardware components, such as, but not limited to, at least one of: microphones and audio capture devices, and software modules capable of processing speech signals. The one or more speech processors 122 are configured to perform operations such as noise reduction, voice activity detection, and signal enhancement to ensure high-quality speech data is used for analysis. The one or more speech processors 122 are further configured to extract one or more acoustic features from the captured speech signals using the one or more machine learning (ML) models. The one or more acoustic features may include, but are not limited to, a pitch, a tone, a speech rhythm, a voice intensity, a spectral energy, formant frequencies, and the like. Such one or more acoustic features are analyzed to infer the one or more physiological indicators, such as, but not limited to, at least one of: emotional variability, fatigue, and stress levels, which are closely associated with speech patterns. For example, variations in the pitch and the tone may indicate the stress levels or the emotional states, while irregularities in speech rhythm or decreased voice intensity may suggest fatigue or cognitive strain.

In an exemplary embodiment, the cognitive computing-based system 102 may be implemented by way of a single device or a combination of multiple devices that may be operatively connected or networked together. The cognitive computing-based system 102 may be implemented in hardware or a suitable combination of hardware and software. In an exemplary embodiment, the one or more image-capturing units 116, the one or more thermal imaging sensors 118, the one or more physiological sensing peripherals 120, and the one or more speech processors 122 may be deployed as a single unit to provide a compact, integrated solution for the multi-modal data acquisition. This single unit may be configured as a unified device or a health monitoring station that incorporates all the necessary components to collect diverse physiological and contextual data from the one or more users in a seamless and efficient manner. The single unit may include structural features, such as an ergonomic design, to ensure the one or more user comfort during the multi-modal data collection. For instance, the single unit may be equipped with a stable mounting mechanism for the one or more image-capturing units 116 and the one or more thermal imaging sensors 118, ensuring consistent alignment and distance from a face of the one or more users. Similarly, the one or more physiological sensing peripherals 120, such as heart rate monitors, pulse oximeters, electrocardiogram (ECG), Non-Contact IR Thermometer (NCIT), and body impedance analyzers, may be integrated into the single unit to enable direct and non-invasive measurement of the one or more physiological parameters.

Though few components and the plurality of subsystems 114 are disclosed in FIGS. 1A-1C, there may be additional components and subsystems which is not shown, such as, but not limited to, ports, routers, repeaters, firewall devices, network devices, the one or more databases 104, network attached storage devices, assets, machinery, instruments, facility equipment, emergency management devices, image capturing devices, any other devices, and combination thereof. The person skilled in the art should not be limiting the components/subsystems shown in FIG. 1A. Although FIG. 1A illustrates the cognitive computing-based system 102, and the one or more communication devices 106 connected to the one or more databases 104, one skilled in the art can envision that the cognitive computing-based system 102, and the one or more communication devices 106 may be connected to several user devices located at various locations and several databases via the one or more communication networks 108.

Those of ordinary skilled in the art will appreciate that the hardware depicted in FIG. 1A may vary for particular implementations. For example, other peripheral devices such as an optical disk drive and the like, the local area network (LAN), the wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the cognitive computing-based system 102 as is unique to the present disclosure or necessary for an understanding of the present disclosure is depicted and described. The remainder of the construction and operation of the cognitive computing-based system 102 may conform to any of the various current implementations and practices that were known in the art.

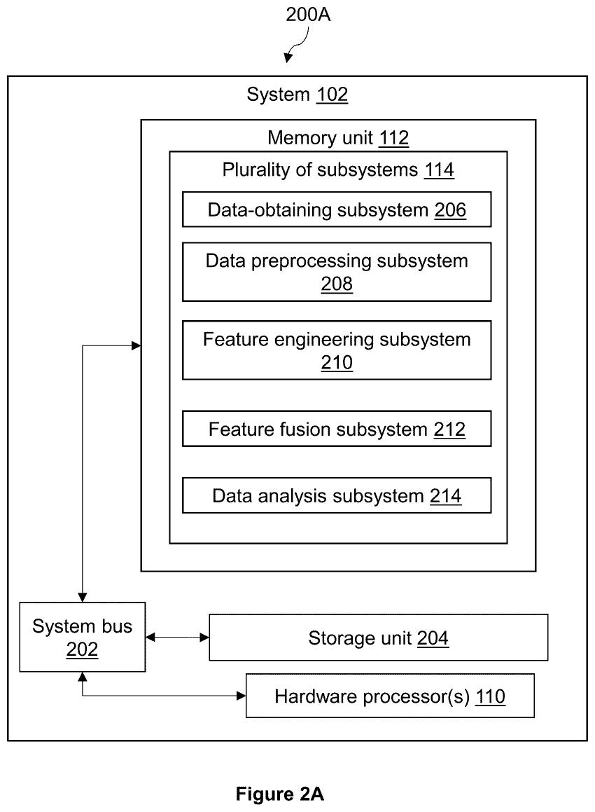

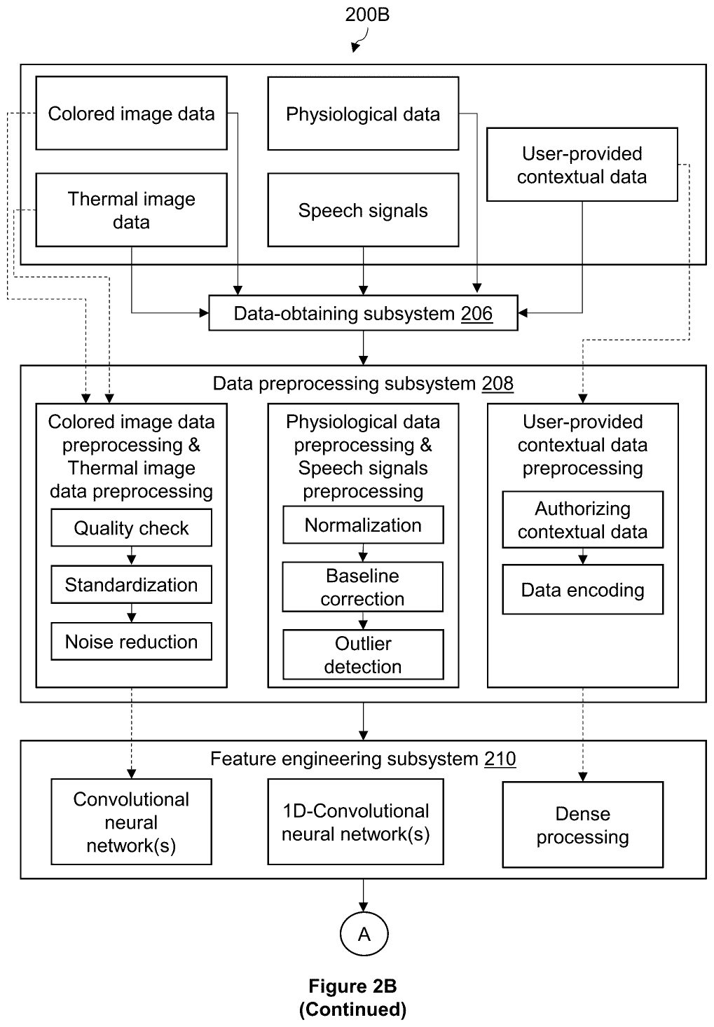

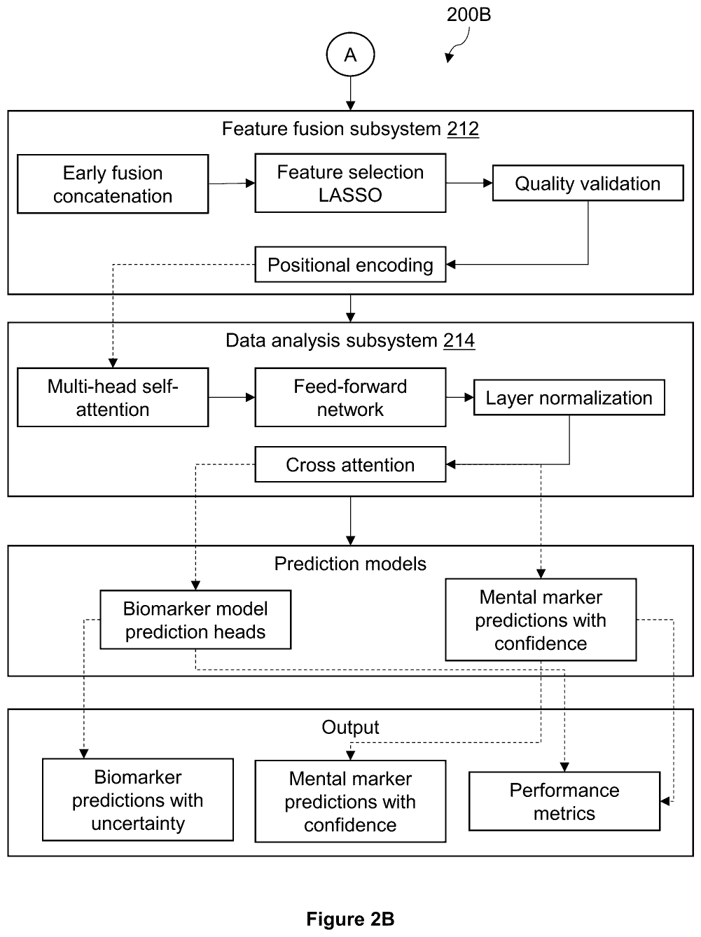

FIG. 2A illustrates an exemplary block diagram representation 200A of the cognitive computing-based system 102 as shown in FIG. 1A, for non-invasive analysis of one or more physiological indicators using assisted transdermal optical imaging, in accordance with an embodiment of the present disclosure.

FIG. 2B illustrates an exemplary flow diagram representation 200B of the cognitive computing-based system 102 as shown in FIG. 2A, for non-invasive analysis of one or more physiological indicators using assisted transdermal optical imaging, in accordance with an embodiment of the present disclosure.

In an exemplary embodiment, the cognitive computing-based system 102 (hereinafter referred to as the system 102) comprises the one or more servers 124, the memory unit 112, and a storage unit 204. The one or more hardware processors 110, the memory unit 112, and the storage unit 204 are communicatively coupled through a system bus 202 or any similar mechanism. The system bus 202 functions as the central conduit for data transfer and communication between the one or more hardware processors 110, the memory unit 112, and the storage unit 204. The system bus 202 facilitates the efficient exchange of information and instructions, enabling the coordinated operation of the system 102. The system bus 202 may be implemented using various technologies, including but not limited to, parallel buses, serial buses, or high-speed data transfer interfaces such as, but not limited to, at least one of a: universal serial bus (USB), peripheral component interconnect express (PCIe), and similar standards.

In an exemplary embodiment, the memory unit 112 is operatively connected to the one or more hardware processors 110. The memory unit 112 comprises the plurality of subsystems 114 in the form of programmable instructions executable by the one or more hardware processors 110. The plurality of subsystems 114 comprises a data-obtaining subsystem 206, a data preprocessing subsystem 208, a feature engineering subsystem 210, a feature fusion subsystem 212, and a data analysis subsystem 214. The one or more hardware processors 110 associated within the one or more servers 124, as used herein, means any type of computational circuit, such as, but not limited to, the microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 110 may also include embedded controllers, such as generic or programmable logic devices or arrays, application-specific integrated circuits, single-chip computers, and the like.

The memory unit 112 may be the non-transitory volatile memory and the non-volatile memory. The memory unit 112 may be coupled to communicate with the one or more hardware processors 110, such as being a computer-readable storage medium. The one or more hardware processors 110 may execute machine-readable instructions and/or source code stored in the memory unit 112. A variety of machine-readable instructions may be stored in and accessed from the memory unit 112. The memory unit 112 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory unit 112 includes the plurality of subsystems 114 stored in the form of machine-readable instructions on any of the above-mentioned storage media and may be in communication with and executed by the one or more hardware processors 110.

The storage unit 204 may be a cloud storage or the one or more databases 104 such as those shown in FIG. 1A. The storage unit 204 may store, but not limited to, recommended course of action sequences dynamically generated by the system 102. These action sequences may comprises on at least one of: analysis of the one or more physiological indicators, temporal trend analysis, detected anomalies or abnormal conditions, user-provided contextual data, feedback from the one or more users, and historical data trends from previous analysis. The dynamically generated action sequences may be used to optimize the evaluation of the system 102, improve response accuracy, enhance accuracy of prediction of the system 102. Additionally, the storage unit 204 may retain previous action sequences for comparison and future reference, enabling continuous refinement of the system 102 over time. The storage unit 204 may be any kind of database such as, but not limited to, relational databases, dedicated databases, dynamic databases, monetized databases, scalable databases, cloud databases, distributed databases, any other databases, and a combination thereof.

In an exemplary embodiment, the data-obtaining subsystem 206 is configured to obtain the multi-modal data from a plurality of sources comprises at least one of: the one or more image-capturing units 116, the one or more thermal imaging sensors 118, the one or more physiological sensing peripherals 120, and one or more users through the one or more communication devices 106. The data-obtaining subsystem 206 serves as a foundational component for acquiring the multi-modal data necessary for the non-invasive analysis of the one or more physiological indicators by the system 102. The obtained multi-modal data is stored in the one or more databases 104 for preprocessing by the system 102 in further steps. The data-obtaining subsystem 206 operates in a robust and adaptive manner to account for variations in data quality, environmental conditions, and device configurations.

The data-obtaining subsystem 206 is configured to communicate with the one or more image-capturing units 116, the one or more thermal imaging sensors 118, the one or more physiological sensing peripherals 120, the one or more speech processors 122 and the one or more users via one or more device-specific application programming interfaces (APIs), standard video interfaces, a universal serial bus (USB), High-definition multimedia interface (HDMI), and the one or more communication networks 108, and the like. Additionally, the data-obtaining subsystem 206 may configured with software layer for seamless multi-modal data integration. The multi-modal data comprises, but not limited to, at least one of: the colored image data, the thermal image data, the physiological data, the speech signals, the user-provided contextual data, and the like.

The colored image data may comprise the one or more red, green, and blue (RGB) images at 1080p resolution and 30 fps. The one or more RGB images are configured with time-stamp to align with other modalities for synchronized processing. The thermal image data comprises thermal images at 640×480 resolution with a sensitivity of <0.05° C. the thermal images may comprises full face region and lower inner eyelid, are specifically targeted to detect temperature patterns, blood flow, and inflammation markers.

In an exemplary embodiment, the data preprocessing subsystem 208 is configured to preprocess the multi-modal data to ensure that the multi-modal data is structured, standardized, and ready for analysis by subsequent subsystems within the system 102. The data preprocessing subsystem 208 handles the unique requirements of each modality, mitigating noise, aligning data, and extracting relevant regions of interest (ROI) to enhance the accuracy and efficiency of the system 102. In the data preprocessing subsystem 208, the colored image data from the one or more image-capturing units 116 is preprocessed based on performing at least one of: a face detection and alignment, a motion compensation, a standardization, an artifact removal, and a region-of-interest (ROI) isolation.

The data preprocessing subsystem 208 is configured to determine the facial regions of the one or more users using one or more object detection models to ensure the region-of-interest (ROI) is consistent across one or more image frames in the colored image data which are sensitive to blood flow and hemoglobin changes. The colored image data is captured at 1080p resolution and 30 fps. The data preprocessing subsystem 208 ensures consistent illumination by checking ambient lighting conditions (500-1000 lux) to minimize shadows or glare. The data preprocessing subsystem 208 is configured to perform at least one of: the motion compensation process and the region-of-interest (ROI) tracking on the colored image data for stabilizing the region-of-interest (ROI) across the one or more image frames. The motion compensation process is configured to correct user movement during data capture by stabilizing the ROI across the one or more image frames. The motion compensation process use techniques such as optical flow or image registration are used to track and realign facial features.

The data preprocessing subsystem 208 is configured to normalize at least one of: brightness, contrast, and color of the colored image data associated to account for differences in lighting conditions. Further, the data preprocessing subsystem 208 is configured to scaling one or more RGB images in the colored image data to a defined dimension for providing uniform input for the one or more machine learning (ML) models. The data preprocessing subsystem 208 is configured to detects and aligns the facial regions in the one or more RGB images using object detection models such as, but not limited to, at least one of: a Haar cascades model, You Only Look Once (YOLO) model, facial landmark detection model, and the like. The object detection models are configured to ensure consistent positioning of the facial regions across the one or more image frames to maintain accuracy in the region-of-interest (ROI) isolation. The Haar cascades model is a machine learning-based approach for object detection, particularly for tasks like face detection. The Haar cascades model employs a cascade of simple features, known as Haar-like features, to efficiently identify objects within an image. These features capture differences in intensity between neighboring regions. The YOLO model, on the other hand, is a state-of-the-art object detection model that divides an image into a grid of cells. Each cell predicts bounding boxes and class probabilities for objects within its region. The YOLO is known for its speed and accuracy, making it suitable for real-time applications. The facial landmark detection model, such as those based on deep learning, are designed to locate specific facial features like eyes, nose, and mouth. The facial landmark detection model may be used to precisely align faces and extract relevant regions of interest.

The data preprocessing subsystem 208 is configured to filter noise caused by uneven lighting, shadows, or reflections to ensure clean colored image data. The data preprocessing subsystem 208 is configured to apply at least one of: histogram equalization and similar methods to normalize brightness and contrast. The histogram equalization method is a technique used to improve the contrast of an image by redistributing the intensity values. The histogram equalization method is configured to calculate the histogram of the image, which shows the frequency of each intensity level. After, cumulative distribution function (CDF) is calculated from the histogram. It represents the cumulative sum of the pixel frequencies up to a certain intensity level. The CDF is normalized to the range [0, 255] for 8-bit images. Each pixel's intensity is mapped to a new intensity based on the normalized CDF. Pixels with low intensity values are mapped to higher intensity values, and vice versa.

The data preprocessing subsystem 208 is configured to preprocess the thermal image data based on performing at least one of: a temperature calibration, a noise reduction, a region-of-interest (ROI) tracking to capture at least one of: temperature patterns, blood flow, and inflammation markers. The data preprocessing subsystem 208 adjusts thermal readings to account for sensor-specific offsets and environmental factors, ensuring accurate temperature measurement within the range of 20° C.-45° C. Applies filtering techniques, such as Gaussian filters or wavelet denoising, to reduce sensor noise and improve thermal image quality. Aligns the one or more thermal image ROIs with corresponding regions in the one or more RGB image data, ensuring spatial consistency across modalities. Extracts temperature distribution and heat gradient information, highlighting inflammation markers and blood flow dynamics from the thermal image data. Further, the data preprocessing subsystem 208 is configured to align the thermal image data with the corresponding-colored image data by mapping the ROI to ensure spatial and temporal consistency across the multi-modal data.

The data preprocessing subsystem 208 is configured to preprocess the physiological data based on performing at least one of: a signal filtering, a baseline correction, a feature extraction for raw signals, a data synchronization with at least one of: timestamps and one or more physiological event markers, outlier detection and exclusion, time normalization, unit standardization, and segmentation of the physiological data at pre-defined intervals. The data preprocessing subsystem 208 is configured to filter raw physiological signals (e.g., ECG, SpO2, or heart rate) to remove noise and artifacts. The baseline correction is applied to account for signal drift or fluctuations caused by movement or inconsistencies. The data preprocessing subsystem 208 is configured preprocess the physiological data using at least one of: a Z-score normalization model, a min-max scaling model, a robust scaling model for at least one of: normalizing the physiological data, standardizing the physiological data, and perform the outlier detection and exclusion. The Z-score normalization model also known as standardization, is a technique used to transform data into a standard normal distribution with a mean of 0 and a standard deviation of 1. This is achieved by subtracting the mean of the data from each data point and then dividing the result by the standard deviation. The Z-score normalization model ensures that the multi-modal data is on the same scale, making it easier to compare and analyze. The Z-score normalization model identify outliers, as they will have significantly larger or smaller Z-scores. The Min-max scaling, also known as normalization, is a technique used to transform data into a specific range, typically between 0 and 1. This is achieved by subtracting the minimum value of the data from each data point and then dividing the result by the range of the data. The robust scaling is a technique that is less sensitive to outliers than traditional scaling methods like the Z-score normalization and the min-max scaling. The robust scaling works by using robust statistical measures like median and interquartile range (IQR) to scale the multi-modal data.

The data preprocessing subsystem 208 is configured to preprocess the user-provided contextual data to perform at least one of: data encoding, authorizing contextual data, and computing composite scores. The data preprocessing subsystem 208 is configured to preprocess the user-provided contextual data using at least one of: a one-hot encoding model, an ordinal encoding model, and a binary encoding model, as the one or more ML models for at least one of: converting into categorical responses, converting into scaled responses, and processing one of: yes and no contextual data. The preprocessing of the user-provided contextual data extracts at least one of: symptom severity scores, risk factor aggregation, temporal change in the contextual data, combined health indicators, and mental state indicators of the one or more users. The data encoding involves encode categorical inputs, such as, but not limited to, at least one of: the stress levels, and lifestyle habits, using the one-hot encoding model, the ordinal encoding model, and the binary encoding model.

In the one-hot encoding model is configured to convert the categorical inputs into binary vectors. For example, user-provided stress levels (“Low,” “Medium,” “High”) are encoded as separate binary features: [1, 0, 0] for “Low,” [0, 1, 0] for “Medium,” and [0, 0, 1] for “High. In the ordinal encoding model is configured to assign ordinal values to ordered categorical data. For instance, lifestyle habits categorized as “Sedentary,” “Moderately Active,” and “Active” are encoded as 1, 2, and 3 respectively. In the binary encoding model, the c data with YES and NO as binary values, with “Yes” mapped to 1 and “No” mapped to 0. For example, user-provided responses to questions such as “Do you take medication for diabetes?” are converted into binary values.

The data preprocessing subsystem 208 is configured to validate and authorize the user-provided contextual data to ensure reliability and consistency. The data preprocessing subsystem 208 ensures that all required fields, such as symptom severity, stress levels, and sleep patterns, are completed by the one or more users. Further, prompts the one or more users to fill in missing data or correct invalid responses. Verifies the authenticity of the user-provided contextual data by checking for logical consistency (e.g., ensuring stress level and sleep quality ratings align with known patterns).

The data preprocessing subsystem 208 is configured to computes composite scores based on user-provided contextual data to generate aggregated metrics that represent overall health indicators or risk factors. The data preprocessing subsystem 208 aggregates user-reported symptoms, such as dizziness, fatigue, or difficulty breathing, into a single severity score based on predefined weighting criteria. For example, “Dizziness=2,” “Fatigue=3,” and “Difficulty Breathing=4” may be combined into a composite score of 3.0. Combines individual health factors (e.g., self-reported lifestyle habits, sleep duration, and pre-existing conditions) into a comprehensive risk score. For instance, a combination of high stress levels, poor sleep quality, and a sedentary lifestyle may yield a high-risk score for cardiovascular disease. Analyzes changes in user-reported data over time to identify trends or patterns. For example, tracking weekly changes in stress levels or sleep quality allows the system 102 to detect worsening conditions or improvements.

In an exemplary embodiment, the feature engineering subsystem 210 is configured to generate one or more multi-modal features based on data obtained and preprocessed by the data preprocessing subsystem 208. The feature engineering subsystem 210 extracts, processes, and derives relevant features from multiple data modalities, ensuring that the generated features are robust, meaningful, and compatible for downstream machine learning analysis. The feature engineering subsystem 210 is configured to extract at least one of: blood flow pattern data, hemoglobin estimation data, tissue perfusion characteristics, and micro-expression detection data from the colored image data. This extraction is based on analyzing at least one of: pixel-level changes, a color spectrum, and subtle facial movements, in the preprocessed colored image data.

The feature engineering subsystem 210 is configured to identify subtle variations in skin tone and texture caused by underlying blood circulation and hemoglobin concentration. The feature engineering subsystem 210 is configured to track temporal changes in pixel intensities across one or more image frames to detect blood flow patterns. The color spectrum is decomposed to identify specific wavelengths corresponding to blood oxygenation and hemoglobin levels. The subtle facial movements, such as micro-expressions, are detected to infer stress, fatigue, and emotional variability. The convolutional neural networks (CNNs), as one or more ML models, are utilized to detect spatial patterns and extract features from the colored image data effectively.

The feature engineering subsystem 210 is further configured to extract at least one of: temperature distribution patterns, inflammation indicator data, and blood flow dynamics from the thermal image data using at least one of: the one or more ML models and one or more image filtering models. The temperature distribution patterns are mapped to detect anomalies, such as localized inflammation or uneven blood flow. The inflammation indicator data is identified by recognizing elevated temperature regions using the one or more ML models trained on thermal data in the one or more labeled datasets. The blood flow dynamics are tracked by analyzing temporal changes in temperature gradients, correlating these changes with cardiovascular activity. Filters such as Gaussian smoothing and edge detection enhance the visibility of thermal patterns.

From the physiological data, the feature engineering subsystem 210 is configured to generate at least one of: time-domain heart rate variability (HRV) features, frequency-domain HRV features, systolic and diastolic blood pressure ratios, respiratory variability, stress and cardiovascular markers, and oxygen saturation fluctuation data. The time-domain HRV features include metrics such as 1D-Convolutional neural networks (CNNs) includes at least one of: a standard deviation of NN intervals (SDNN) and root mean square of successive differences (RMSSD), which quantify heartbeat interval variability. Frequency-domain HRV features analyze the power spectral density of heart rate signals, extracting first-frequency (Low frequency (LF)) and second-frequency (high frequency (HF)) components to evaluate autonomic nervous system activity. Ratios such as LF/HF are used to assess stress levels and cardiovascular health. The feature engineering subsystem 210 also computes systolic and diastolic blood pressure ratios and tracks respiratory variability and oxygen saturation levels for insights into cardiovascular and respiratory functions.

In addition to processing the physiological data, the feature engineering subsystem 210 determines at least one of: encoded features and derived metrics from the user-provided contextual data using a dense processing. The encoded features are generated by converting categorical inputs, such as stress levels and lifestyle habits, into numerical representations using techniques like the one-hot encoding, the ordinal encoding, and the binary encoding. The derived metrics, such as combined health indicators, symptom severity scores, and mental state indicators, are computed by aggregating multiple contextual inputs. For example, stress levels, sleep quality, and symptoms may be combined to generate a “fatigue risk score” or “anxiety index.”

The feature engineering subsystem 210 integrates features from all modalities into a unified feature set, ensuring that the one or more multi-modal features from the colored image data, the thermal image data, the physiological data, and the contextual data are complementary and non-redundant. Techniques such as cross-modal feature learning and attention-based models are applied to identify relationships between features from the multi-model data. For instance, thermal patterns of inflammation may be correlated with blood flow patterns from colored image data to improve the accuracy of stress-level predictions.

The feature engineering subsystem 210 leverages the one or more CNNs as the one or more ML models for the one or more multi-modal features extraction. The one or more CNNs process spatial features from the colored image data, the thermal image data and generate advanced HRV features, such as LF/HF ratios, to evaluate autonomic nervous system activity and cardiovascular markers. By extracting and synthesizing multi-modal features, the feature engineering subsystem 210 enables comprehensive and precise analysis of the one or more physiological indicators.

In an exemplary embodiment, the feature fusion subsystem 212 is configured to synchronize and integrate multi-modal data obtained from a plurality of sources to generate unified features representation data for the one or more ML model analysis. The synchronization of the multi-modal data is achieved using at least one of: the timestamps and the one or more physiological event markers as anchor points. The timestamps ensure temporal alignment across the multi-modal data streams from the modalities such as the colored image data, the thermal image data, the physiological data, the speech signals, and the user-provided contextual data, while physiological event markers, such as heartbeats and respiratory cycles, provide precise biological reference points for synchronization. Once synchronized, the feature fusion subsystem 212 integrates the generated one or more multi-modal features into unified feature representation data that captures the collective information from the various data modalities.

To ensure the quality and reliability of the unified feature representation, the feature fusion subsystem 212 assigns one or more domain-specific constraints to the integrated one or more multi-modal features. The one or more domain-specific constraints are configured to alleviate one or more physiologically inapt combinations that may arise from noise, misalignment, and inconsistencies across the multi-modal data. For example, one or more domain-specific constraints may enforce logical coherence between blood flow dynamics derived from thermal images and blood pressure ratios extracted from the physiological data.

The generation of the unified feature representation data involves the use of advanced methodologies, including at least one of: modality-specific attention procedures, a cross-modal relationship learning model, and adaptive feature importance weighting procedures. The modality-specific attention procedures are employed to focus on the most informative aspects of each multi-modal feature of the one or more multi-modal features, filtering out irrelevant or redundant information. The cross-modal relationship learning models identify and leverage interdependencies between different data modalities, such as the correlation between temperature patterns in thermal data and stress indicators in contextual data. Adaptive feature importance weighting procedures dynamically assign weights to each multi-modal feature of the one or more multi-modal features based on its reliability and relevance for predicting physiological indicators. For instance, the one or more multi-modal features with higher predictive reliability, such as accurately captured HRV metrics, are given greater importance in the unified representation. The unified feature representation data generated by the feature fusion subsystem 212 ensures a comprehensive and coherent input for downstream the one or more ML models analysis.

In an exemplary embodiment, the data analysis subsystem 214 is configured with the one or more ML models to perform non-invasive analysis on the unified features representation data based on generating a data correlation with one or more labeled datasets to predict the one or more physiological indicators. The one or more physiological indicators comprise, but not limited to, at least one of: hemoglobin concentration, glycated hemoglobin (HbA1c), blood pressure, heart rate variability (HRV), blood oxygen saturation (SpO₂), blood glucose, respiratory rate, stress levels, cognitive load, fatigue, emotional variability, and the like. The one or more ML models comprise at least one of: an XGBoost Regressor, the one or more convolutional neural networks (CNNs) with a long short-term memory (LSTM) hybrid, a random forest, a bidirectional long short-term memory (BiLSTM), a transformer-convolutional neural network (CNN), recurrent neural networks (RNNs), light gradient-boosting machine (LightGBM), a transformer-based multi-task model, deep neural network, gradient boosting, neural networks, a k-means clustering, a hierarchical clustering and support vector machines (SVMs)

The data analysis subsystem 214 is further configured to detect one or more temporal trends in the unified features representation data, including at least one of: changes in the one or more physiological indicators over time, recurring patterns in the physiological data, and one or more abnormal health conditions. Additionally, the data analysis subsystem 214 generates at least one of: the one or more numerical predictive insights, the visual representations data, the one or more categorical predictive insights, and the recommendation data of the one or more physiological indicators, based on the non-invasive analysis of the one or more physiological indicators.

The data analysis subsystem 214 is configured to select the one or more ML models based on the type of physiological indicators being predicted within the one or more physiological indicators. The one or more ML models include at least one of: supervised learning models for labeled data, unsupervised learning models for unlabeled data, and deep learning models for complex multi-modal data analysis. For supervised learning, the data analysis subsystem 214 uses regression models, such as the XGBoost Regressor and the random forest, to determine continuous variables like hemoglobin concentration and glycated hemoglobin (HbA1c). The classification models, such as the support vector machines (SVMs) and the one or more convolutional neural networks (CNNs), are employed to predict categorical outputs, including stress levels and inflammation markers.

For analyzing unlabeled data within the multi-modal data, the data analysis subsystem 214 utilizes unsupervised learning models, including clustering models such as k-means clustering and hierarchical clustering. The unsupervised learning models identify patterns within the unified features representation data of the one or more users, based on the one or more multi-modal features extracted from the multi-modal data. This enables the system 102 to group the one or more users with similar physiological profiles and detect novel patterns that may indicate emerging conditions.

The data analysis subsystem 214 is also configured to employ deep learning models, including recurrent neural networks (RNNs), transformer-convolutional neural networks (CNNs), and long short-term memory (LSTM) hybrids, for complex data analysis. These one or more ML models analyze at least one of: blood flow patterns, hemoglobin estimation data, tissue perfusion characteristics, and micro-expression detection data in the colored image data. The deep learning models enable the system 102 to capture temporal dependencies and intricate relationships within the data, enhancing the accuracy of physiological indicator predictions.

In an exemplary embodiment, a temporal analysis is applied to time-series data to extract and analyze patterns in the physiological data such as the heart rate variability (HRV) and blood flow dynamics. The temporal analysis leverages advanced techniques to identify trends, fluctuations, and significant events in the data over time. The RNNs and LSTM models are employed to capture sequential dependencies in physiological features, such as HRV fluctuations or thermal temperature trends. These RNNs and LSTM models are specifically configured to process time-series data and retain information from previous time steps, allowing the system 102 to understand how the one or more physiological indicators evolve over time. Additionally, attention mechanisms are used to pinpoint critical time points or physiological events that have a significant impact on the predictions, ensuring that the most relevant data is prioritized in the analysis. A sliding window analysis is also utilized to segment continuous data streams into overlapping windows, enabling localized and granular analysis of time-dependent features, which is particularly useful for detecting short-term variations or anomalies in physiological data.

By integrating the one or more machine learning techniques, the data analysis subsystem 214 provides a comprehensive and adaptive framework for analyzing unified features representation data. It not only predicts the one or more physiological indicators with high accuracy but also generates actionable insights, such as recurring trends and personalized recommendations, ensuring that the system 102 effectively supports non-invasive health monitoring and decision-making.

In an exemplary embodiment, the one or more ML models are trained using at least one of: one or more loss function models, stochastic gradient descent (SGD), Adaptive Moment Estimation (Adam), Root Mean Square Propagation (RMSprop), hyperparameter tuning models, and the like. These methods are employed to optimize the one or more ML models for tasks such as continuous variable prediction, classification of the multi-modal data, and alleviating the one or more physiologically inapt combinations within the unified feature representation data. The one or more ML models are further configured to adaptively retrain on real-time acquired multi-modal data to optimize prediction accuracy and address data drift caused by variations in at least one of the following factors: demographic information, environmental conditions, and one or more sensor settings. Additionally, the system 102 incorporates one or more feedback loops from the one or more users to refine the predictions and outputs of the one or more ML models over time, ensuring continuous improvement and personalization of the system's 102 performance.

The one or more ML model training and validation involves two key components: the training process and validation strategies. In the training process, the one or more ML models utilize features engineered from multi-modal data. The one or more multi-modal features include RGB-based features, such as blood flow patterns and hemoglobin concentration; thermal-based features, such as temperature asymmetry and stress indicators; and clinical parameters and user-reported metrics. Specific loss functions are applied based on the type of physiological indicator being predicted: Huber Loss is used for HbA1c prediction to handle robust regression, the mean squared error (MSE) is used for hemoglobin estimation, and binary cross-entropy (BCE) is applied for categorical outputs like mental markers and stress levels. The validation is performed using 5-fold cross-validation to ensure robustness across data subsets, while temporal validation is employed to address time-sensitive health indicators and ensure the models' applicability to real-world scenarios.

In an exemplary embodiment, the retraining the one or more ML models are implemented to evolve data and maintain high prediction accuracy. The retraining is triggered when multi-modal data drift is detected, the one or more ML models performance drops by more than 5%, or periodically, such as every three months. The retraining methods include incremental updates, which allow the one or more ML model models to adapt to new data while retaining existing knowledge, and full model retraining for significant performance improvements when the multi-modal data distribution changes substantially.

In an exemplary embodiment, one or more calibration techniques and one or more quality assurance mechanisms are employed to maintain the reliability and accuracy of the system 102. The one or more calibration techniques include image calibration, which standardizes resolution and lighting conditions, and aligns colored image data and thermal image features for effective feature fusion. Clinical calibration is performed by normalizing clinical data (e.g., using the Z-score normalization) and making adjustments based on user demographics, such as age or gender. Quality Checks ensure data integrity and model reliability. Input validation detects missing values, sensor malfunctions, or environmental artifacts that could compromise data quality. The one or more ML models validation involves predictive confidence scoring and comparisons with clinical benchmarks to ensure medical-grade accuracy, thereby making the system 102 suitable for non-invasive physiological analysis.

In an exemplary embodiment, a post-prediction refinement is performed to enhance the interpretability and reliability of the one or more ML model's outputs. This process includes assigning confidence scores to predictions, which are calculated based on the probabilities generated by the one or more ML models. Confidence scoring provides the one or more users with an indication of the certainty associated with each prediction, improving trust in the system's 102 outputs. Domain-based corrections are also applied to ensure that predictions are physiologically plausible. For example, if a predicted hemoglobin concentration falls outside the expected human range, domain-specific rules are used to adjust the value to a more realistic level. This ensures that the outputs remain accurate and medically relevant, even in cases where the raw model predictions may deviate due to noise or outliers in the data.

The results generated by the one or more ML models are then formatted and presented for the one or more users interpretation or downstream use. These outputs can be categorized into three primary types: the one or more numerical prediction insights, the one or more categorical predictive insights, and the visual representations data. The one or more numerical prediction insights provide precise quantitative results, such as “Hemoglobin concentration=13.5 g/dL” or “HRV=42 ms,” enabling detailed analysis of specific physiological indicators. The one or more categorical predictive insights assign qualitative labels to the multi-modal data, such as “Stress level=Moderate” or “Risk level=Low,” offering a clear summary of the system's 102 assessments. The visual representations data, such as heatmaps highlighting inflammation areas or time-series graphs illustrating HRV trends, provide intuitive and easily interpretable visual insights into the physiological data. These outputs are configured to be accessible and actionable, ensuring that the one or more users, whether they are clinicians or end-users, may derive meaningful insights from the system's 102 analysis.

For instance, the Electrocardiogram (ECG) flags and ECG-derived features are input to the one or more ML models as the one or more labeled datasets. The ECG flags are depicted in Table 1

TABLE 1

| ECG Flags | Description |
|---|---|
| 0 Asystole (No Heartbeat) | Absence of heartbeat. |
| 1 Ventricular Fibrillation (VF) | Abnormal, rapid heart rhythm |
| | originating in the ventricles. |
| 2 Ventricular Tachycardia | Rapid heart rate originating from |
| | the ventricles. |
| 3 R on T (Ventricular Ectopic Beat During | Ventricular ectopic beat during a |
| Vulnerable Period) | vulnerable period. |
| 4 Multiple Ventricular Premature Beats | Multiple premature beats |
| (VPB) | originating from the ventricles. |
| 5 Nonsustained Ventricular Tachycardia | Ventricular tachycardia that lasts |
| | less than 30 seconds. |
| 6 Coupled Ventricular Premature Beats | Paired premature beats |
| | originating from the ventricles. |
| 7 Accidental Ventricular Premature Beat | A premature beat originating |
| | from the ventricles that occurs |
| | by accident. |
| 8 Bigeminy (Every Other Beat is | A pattern where every other beat |
| Premature) | is premature. |
| 9 Trigeminy (Every Third Beat is | A pattern where every third beat |
| Premature) | is premature. |
| 10 Supraventricular Tachycardia | Rapid heart rate originating |
| | above the ventricles. |
| 11 Supraventricular Bradycardia | Slow heart rate originating above |
| | the ventricles. |
| 12 Multiform Ventricular Premature Beats | Multiple types of premature |
| | ventricular beats. |
| 13 Pace Not Captured (Pacemaker Issue) | A condition where a pacemaker |
| | fails to capture and stimulate the |
| | heart. |
| 14 Pacer Not Paced (Pacemaker Issue) | A condition where the |
| | pacemaker fails to generate |
| | pacing signals. |
| 15 Irregular Rhythm | Heart rhythm with irregular |
| | intervals. |
| 16 Missed Beat (Dropped Beat) | A beat that was missed, resulting |
| | in a dropped interval. |
| 17 Ventricular Rhythm (Abnormal Heart | Abnormal rhythm originating |
| Rhythm) | from the ventricles. |
| 18 Ventricular Bradycardia (Slow Heart | Slow heart rate originating from |
| Rate) | the ventricles. |
| 19 Extreme Tachycardia (Very Fast Heart | Extremely fast heart rate. |
| Rate) | |
| 20 Extreme Bradycardia (Very Slow Heart | Extremely slow heart rate. |
| Rate) | |
| 22 Normal Sinus Rhythm (Healthy Heart | Normal heart rhythm originating |
| Rhythm) | from the sinus node. |
| 24 Too Much Noise (ECG Interference) | ECG data affected by noise or |
| | interference. |
| 27 ST Elevation (Possible Heart Attack) | Elevation of the ST segment, |
| | indicative of myocardial |
| | infarction (heart attack). |
| 28 ST Depression (Possible Heart | Depression of the ST segment, |
| Ischemia) | indicative of heart ischemia. |
| 29 Atrial Fibrillation (Irregular Heart | An irregular and rapid heart |
| Rhythm) | rhythm originating from the |
| | atria. |

The ECG flags are categorical values that represent various abnormalities or conditions identified in ECG signals. Each flag corresponds to a specific condition such as arrhythmias (e.g., Ventricular Fibrillation, Atrial Fibrillation), conduction problems (e.g., Bundle Branch Block), or other heart rhythm disorders (Bigeminy, Trigeminy). These ECG flags may be captured during real-time ECG monitoring and provide immediate diagnostic insights, making them valuable for early detection of critical heart conditions.

The system 102 derived the ECG derived Features input into the one or more ML models as the one or more labeled datasets as depicted in Table 2

TABLE 2

| | | | Normal Range / |
|---|---|---|---|
| | | Measurement | Clinical |
| Feature Type | Metric | Description | Significance |
| 1. Heart Rate | A. Time Domain | | |
| Variability | Measurements | | |
| (HRV) | | | |
| Metrics | | | |
| SDNN | Standard deviation of | Overall measure of | Normal Range: 20- |
| (Standard | normal-to-normal (NN) | autonomic nervous system | 200 ms (24-hour |
| Deviation of | intervals in milliseconds | function. Lower values | recording) |
| NN Intervals) | | indicate reduced HRV. | Clinical |
| | | | Significance: Values < |
| | | | 50 ms require |
| | | | clinical attention. |
| | | | Used for stress and |
| | | | recovery assessment. |
| RMSSD | Square root of the mean | Reflects parasympathetic | Normal Range: 15- |
| (Root Mean | squared differences | activity. Higher values | 40 ms |
| Square of | between successive NN | indicate better cardiac | Clinical |
| Successive | intervals | health. | Significance: Used |
| Differences) | | | for short-term |
| | | | variability |
| | | | assessment. |
| pNN50 | Percentage of successive | Marker of | Normal Range: 3- |
| | NN intervals differing by | parasympathetic activity. | 15% |
| | more than 50 ms | Decreased in stress and | |
| | | certain cardiac conditions. | |
| B. Frequency | Low Frequency (LF) | Frequency range | Normal Range: 30- |
| Domain | | reflecting both | 50% of total power |
| Measurements | | sympathetic and | |
| | | parasympathetic activity. | |
| LF/HF Ratio | Ratio between LF and | Indicates sympathovagal | Normal Range: 0.5- |
| | HF power | balance, with values | 2.0 |
| | | representing autonomic | |
| | | nervous system activity. | |
| High | Reflects parasympathetic | Frequency range | Normal Range: 15- |
| Frequency | activity | indicative of | 40% of total power |
| (HF) | | parasympathetic | |
| | | influence. | |
| 2. Interval | QT Interval | Time from Q wave start to | Male Normal Range: |
| Measurements | | T wave end | 350-450 ms |
| | | | Female Normal |
| | | | Range: 360-460 ms |
| | | | Clinical |
| | | | Significance: |
| | | | Prolonged QTc |
| | | | (>470 ms for |
| | | | females, >450 ms |
| | | | for males) increases |
| | | | arrhythmia risk. |
| | PR Interval | Time from P wave start to | Normal Range: 120- |
| | | QRS complex | 200 ms |
| | | | Clinical |
| | | | Significance: AV |
| | | | conduction |
| | | | abnormalities, first- |
| | | | degree AV block for >200 ms. |
| | QRS Duration | Width of QRS complex | Normal Range: 60- |
| | | (time taken for ventricular | 100 ms |
| | | depolarization) | Clinical |
| | | | Significance: Bundle |
| | | | branch block or |
| | | | ventricular |
| | | | conduction delay |
| | | | if >120 ms. |
| 3. Waveform | ST Segment Analysis | Segment between QRS | Normal Range: ±0.1 |
| Analysis | | end and T wave beginning | mV from baseline |
| | | | Clinical |
| | | | Significance: |
| | | | Elevation indicates |
| | | | possible myocardial |
| | | | infarction, depression |
| | | | indicates ischemia. |
| | T-wave Morphology | T-wave amplitude, | Clinical |
| | | symmetry, area, and | Significance: |
| | | alternans | Inversion may |
| | | | indicate ischemia, |
| | | | alternans indicates |
| | | | electrical instability, |
| | | | peaked T-waves |
| | | | indicate |
| | | | hyperkalemia. |
| 4. R-R | Basic Statistics | Mean R-R, R-R interval | Used for evaluating |
| Interval | Advanced Metrics | standard deviation, R-R | cardiac rhythm and |
| Analysis | | interval histogram | variability. |
| | | Short-term variability (5 | Used for circadian |
| | | min windows), long-term | pattern analysis and |
| | | variability (24-hour | assessing autonomic |
| | | trends) | nervous system |
| | | | function. |
| | Heart Rate Turbulence | Measures the deceleration | |
| | | of heart rate following a | |
| | | premature beat | |
| | Deceleration Capacity | Measures the heart's | |
| | | ability to slow after a | |
| | | premature beat | |
| | Acceleration Capacity | Measures the heart's | |
| | | ability to increase rate | |
| | | after stress | |

The ECG-derived features are critical for understanding heart function, detecting abnormalities, and evaluating a user's cardiovascular health. They are essential inputs for the one or more ML models in the data analysis subsystem 214, providing the system 102 with the ability to make accurate predictions regarding a user's physiological state, and are invaluable for non-invasive health monitoring.

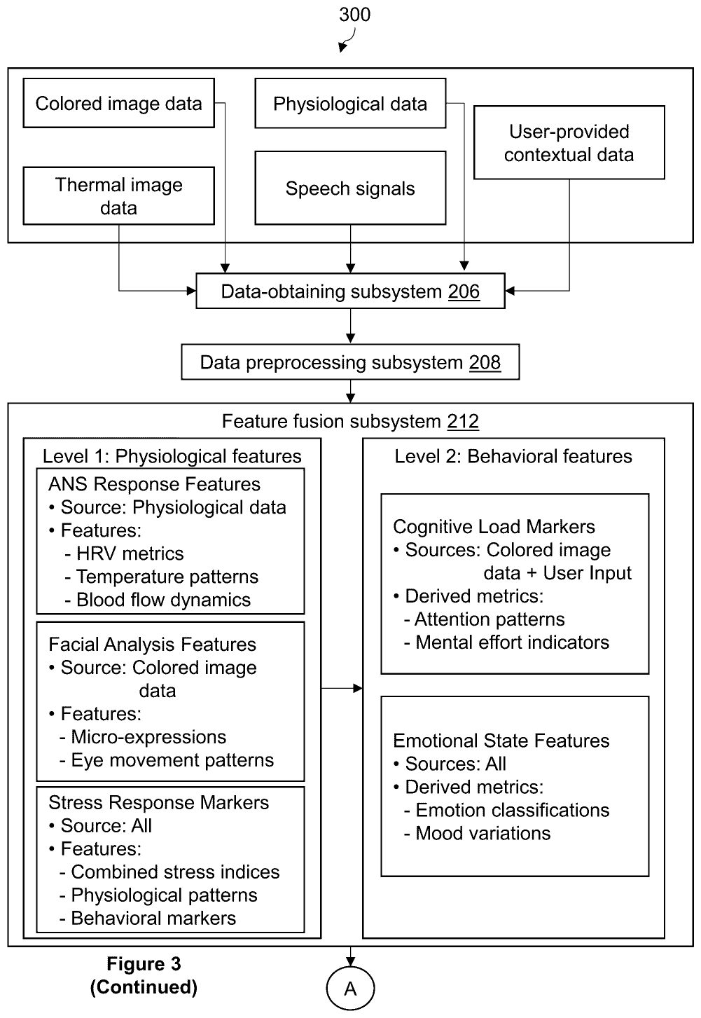

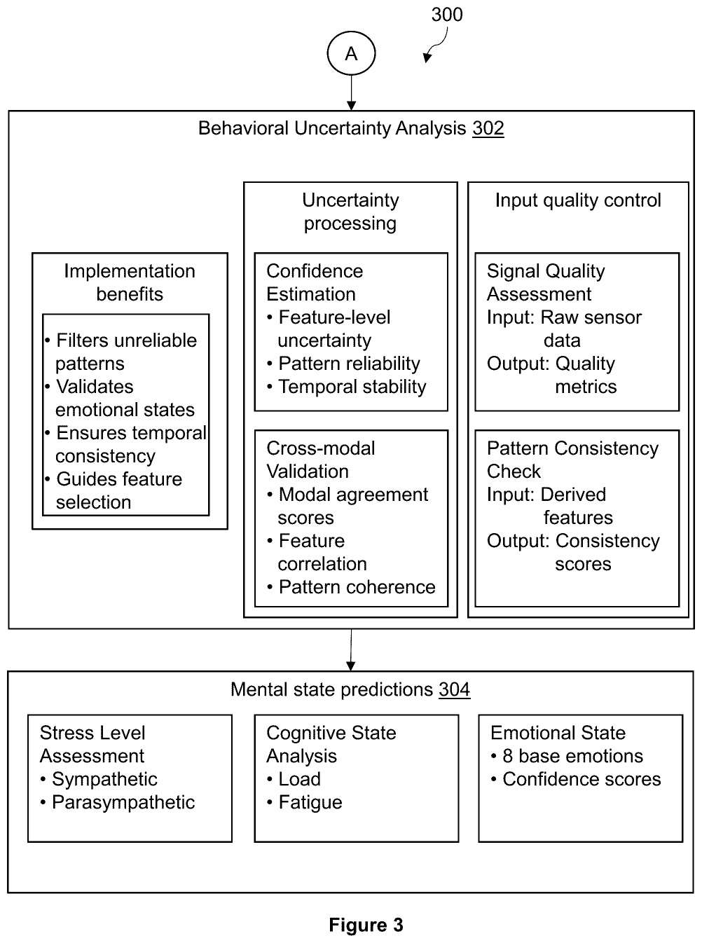

FIG. 3 illustrates an exemplary mental-marker flow architecture 300, in accordance with an embodiment of the present disclosure.

In an exemplary embodiment, the data-obtaining subsystem 206 is configured to obtain the multi-modal data i.e., at least one of: the colored image data, the thermal image data, the physiological data, the speech signals, and the user-provided contextual data. The multi-modal data is input into the data pre-processing subsystem 208 for pre-processing of the multi-modal data. Further, the pre-processed multi-modal data is transferred to the feature engineering subsystem 210 for extracting the mental-markers. The feature engineering subsystem 210 is configured to divide the pre-processed multi-modal data into two main levels: physiological features (Level 1) and behavioral features (Level 2), each focusing on specific aspects of the data.

The physiological features (Level 1) comprises autonomic nervous system (ANS) response features, facial analysis features, and stress response markers. The ANS response features derived from the physiological data and thermal image data, these features include HRV metrics, temperature patterns, and blood flow dynamics. These features are crucial for understanding the user's autonomic nervous system (ANS) function, which is impacted by stress, anxiety, and other mental states. Next, the facial analysis features extracted from colored image data, these features include micro-expressions data, and eye movement patterns. They provide insight into the emotional states of the one or more user, detecting subtle changes in facial expressions that are associated with stress, fatigue, and cognitive load. Further, the stress response markers derived from the plurality of sources, these features combine physiological patterns (like HRV and blood flow) and behavioral markers (such as facial expressions and user input) to assess the overall stress response of the one or more users.

The behavioral features (Level 2) comprises cognitive load markers and emotional state features. The cognitive load markers are derived from the colored image data and the user-provided contextual data. The derived metrics of the cognitive load markers include attention patterns, and mental effort indicators, which provide insights into the cognitive load the one or more users are experiencing, often linked to mental fatigue or concentration. Furthermore, the emotional state features are generated from the plurality of sources, and involve emotion classifications, and mood variations. These emotional state features give a detailed view of the user's emotional state, such as levels of happiness, stress, or anxiety, and how these emotions change over time.

Once the one or more multi-modal features are extracted a behavioral uncertainty analysis module 302 is employed to ensure the accuracy and reliability of the data being analyzed. The behavioral uncertainty analysis module 302 consists of two key components: input quality control and uncertainty estimation. In input quality control, a) signal quality assessment and b) pattern consistency check steps are disclosed. The signal quality assessment involves assessing the raw multi-modal data to ensure it is of high quality and free from noise or artifacts. The system 102 assigns quality metrics to evaluate the reliability of the obtained multi-modal data. In the pattern consistency check step examines the consistency of the derived one or more multi-modal features over time, ensuring that the patterns extracted from the data (e.g., facial expressions, HRV) remain stable and reliable.

In the uncertainty estimation, a) a confidence estimation, and b) a cross-modal validation, are disclosed. In the confidence estimation, feature-level uncertainty is assessed, and the reliability of each extracted feature within the one or more multi-modal features is calculated. It evaluates how stable and reliable each feature of the one or more multi-modal features is over time, providing a confidence score that helps refine predictions. In the cross-modal validation the features from multi-modal data (e.g., facial expressions, HRV, user input) by comparing and cross-referencing them to ensure coherence and accuracy. The cross-modal validation ensures that the patterns identified in different data types align with one another, increasing the reliability of predictions.

The primary benefits of the behavioral uncertainty analysis module 302 is ensure that only consistent and valid the one or more multi-modal features are used in prediction. Guarantees that the emotional states predicted by the system 102 are grounded in reliable data. Authenticates that the multi-modal data over time shows stable patterns, allowing for accurate time-based predictions. Assists in choosing the most reliable one or more multi-modal features for further analysis and prediction.

Further, the system 102 generates predictions regarding the user's mental state using the processed and validated data using a mental state prediction module 304. The mental state prediction module 304 is configured to categorize the predictions into three types of outputs: a stress level assessment, a cognitive state analysis, and an emotional state. The stress level assessment predicts the stress levels of the one or more users, broken down into sympathetic and parasympathetic responses. It provides a comprehensive view of how stress impacts the user's physiological state. The cognitive state analysis assesses cognitive load and fatigue, giving insights into the user's cognitive performance and mental effort. The emotional state provides emotion classifications across eight basic emotions (such as happiness, sadness, anxiety) and assigns confidence scores to each classification. The emotional state tracks emotional state variations over time and provides a real-time analysis of the user's emotional well-being.

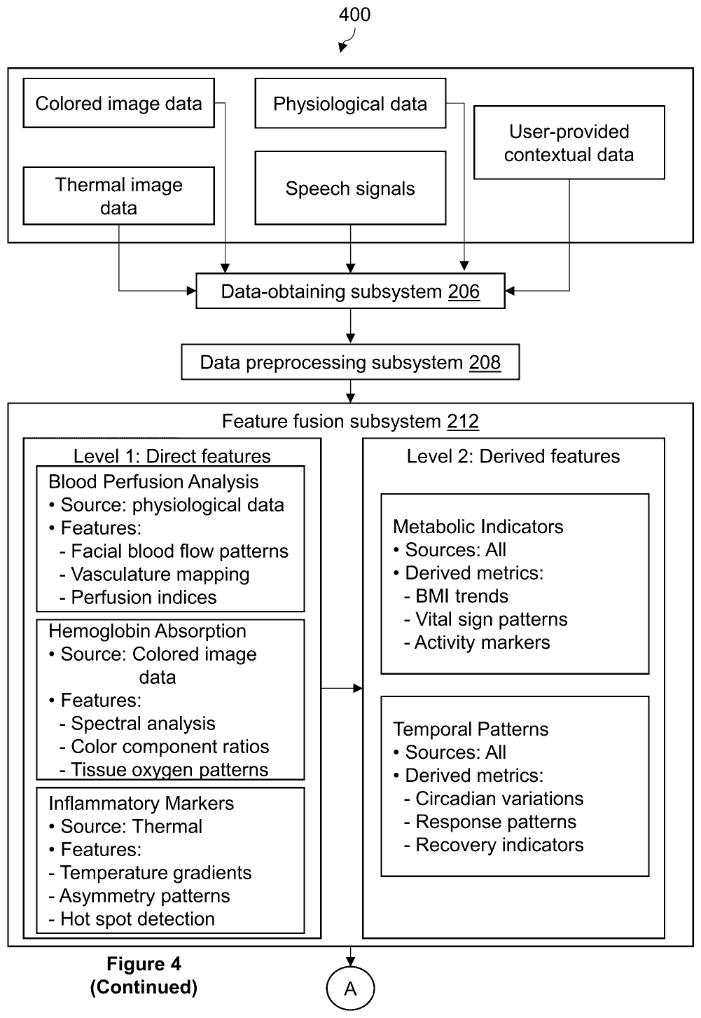

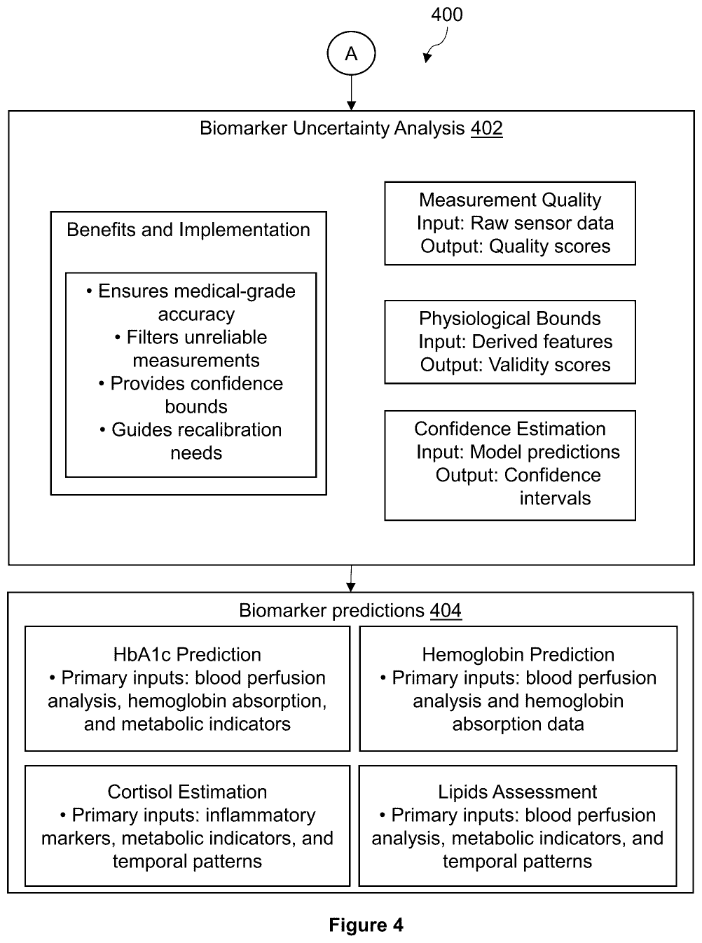

FIG. 4 illustrates an exemplary bio-marker flow architecture 400, in accordance with an embodiment of the present disclosure.

In an exemplary embodiment, the data-obtaining subsystem 206 is configured to obtain the multi-modal data i.e., at least one of: the colored image data, the thermal image data, the physiological data, the speech signals, and the user-provided contextual data. The multi-modal data is input into the data pre-processing subsystem 208 for pre-processing of the multi-modal data. Further, the pre-processed multi-modal data is transferred to the feature engineering subsystem 210 for extracting the bio-markers. The feature engineering subsystem 210 is configured to generate the one or more multi-modal features and categorized into direct features (Level 1) and derived features (Level 2), based on their complexity and the data they are derived from.

In the direct features (Level 1) comprise: a) blood perfusion analysis, b) hemoglobin absorption, and c) inflammatory markers are analyzed. The blood perfusion analysis is derived from both the colored image data and the thermal image data. The blood perfusion analysis includes analysis of facial blood flow patterns, vasculature mapping, and perfusion indices. The one or more multi-modal features are vital for understanding the distribution of blood and oxygen in tissues, which directly relates to hemoglobin levels and overall vascular health. The hemoglobin absorption analysis is processed by using the colored image data. This feature assesses spectral analysis, color component ratios, and tissue oxygen patterns. The system 102 provides crucial information on the oxygen-carrying capacity of the blood and is directly linked to hemoglobin levels. The inflammatory markers extracted from the thermal image data, this feature includes temperature gradients, asymmetry patterns, and hot spot detection. The one or more physiological indicators are essential for detecting signs of inflammation, which may influence biomarkers such as stress levels, cognitive load, fatigue, and emotional variability.

In the derived features (Level 2) comprise: metabolic indicators, and the one or more temporal trends are disclosed. The metabolic indicators are extracted by combining the multi-modal data such as BMI trends, vital sign patterns, and activity markers. The metabolic indicators are useful for evaluating the metabolic health of the one or more users and may contribute to the prediction of biomarkers like lipids and hemoglobin. The one or more temporal trends are derived from the multi-modal data, capturing circadian variations, response patterns, and recovery indicators. The one or more temporal trends are important for understanding how biomarkers fluctuate over time, particularly in relation to lifestyle factors, and are essential for assessing biomarkers like cortisol.

Once the one or more multi-modal features are extracted a biomarker uncertainty analysis module 402 is employed to ensure the accuracy and reliability of the data being analyzed for prediction are of high quality and consistent. The biomarker uncertainty analysis module 402 is configured with a measurement quality, physiological bounds, and a confidence estimation. In the measurement quality the biomarker uncertainty analysis module 402 is configured to identify potential issues such as noise, sensor malfunctions, and environmental artifacts that may affect the multi-modal data quality. The system 102 assigns quality scores to each data source in the plurality of sources, ensuring that only reliable data is used for feature extraction and prediction. Once the one or more multi-modal features are derived from the multi-modal data, the next step is to ensure that they are physiologically plausible. This is done by comparing the extracted one or more multi-modal features such as blood perfusion patterns, hemoglobin absorption rates, and inflammatory markers, to established physiological bounds or thresholds. If a feature falls outside the expected range, it is flagged as invalid or unreliable, and a validity score is assigned to each feature to assess its accuracy and relevance to the prediction task. After the one or more multi-modal features are processed and validated, the system 102 evaluates the uncertainty in its predictions. This is done by calculating confidence intervals for the one or more ML model's predictions, such as HbA1c, hemoglobin, cortisol, and lipids. The confidence estimation process provides a measure of how certain the system 102 is about each prediction, allowing the one or more users to make more informed decisions based on the level of uncertainty associated with the result.

In an exemplary embodiment, the biomarker uncertainty analysis module 402 provides several benefits to ensure medical-grade accuracy. The biomarker uncertainty analysis module 402 ensures that only high-quality data is used for further analysis, eliminating unreliable or noisy measurements that may lead to inaccurate predictions. By quantifying uncertainty, the system 102 offers confidence bounds for its predictions, giving the one or more users a clearer understanding of the reliability of the results. The system 102 may identify when recalibration of the system 102 itself is required to maintain accurate measurements, ensuring continuous performance and accuracy over time.

Finally, a biomarker prediction module 404 is configured to provide actionable health insights based on the processed and validated data. The biomarker prediction module 404 is configured to provide predictions focus on key biomarkers that offer valuable information about the user's physiological state. The system 102 predicts the HbA1c level, a marker for long-term blood glucose control, based on blood perfusion analysis, hemoglobin absorption, and metabolic indicators. The predicted range for HbA1c provides an indication of the user's average blood glucose levels over a prolonged period. The prediction of hemoglobin levels, which indicate the oxygen-carrying capacity of the blood, is based on the blood perfusion analysis and the hemoglobin absorption data. The predicted range for hemoglobin helping to assess the user's cardiovascular and overall health.

The system 102 estimates stress levels, cognitive load, fatigue, emotional variability, and a key stress hormone, by analyzing inflammatory markers, metabolic indicators, and temporal patterns. The predicted range for cortisol is between 0-50 mcg/dL, providing insight into the user's stress levels and adrenal function. The system 102 assesses the user's lipid levels, including cholesterol and triglycerides, which are critical for cardiovascular health. This prediction is based on blood perfusion analysis, metabolic indicators, and temporal patterns. The lipid levels are measured across multiple ranges depending on the specific lipid being assessed, providing detailed information about the user's cardiovascular risk. This process of uncertainty analysis, combined with the robust predictions for biomarkers like HbA1c, hemoglobin, cortisol, and lipids, ensures the system 102 provides clinically relevant, high-quality health insights that users and healthcare professionals can rely on for informed decision-making.

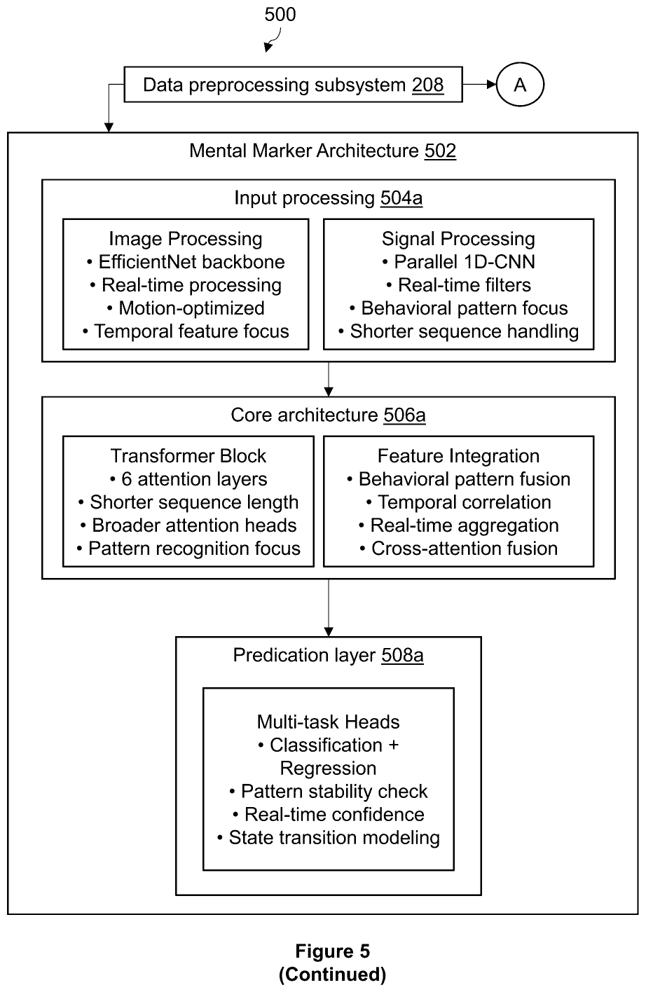

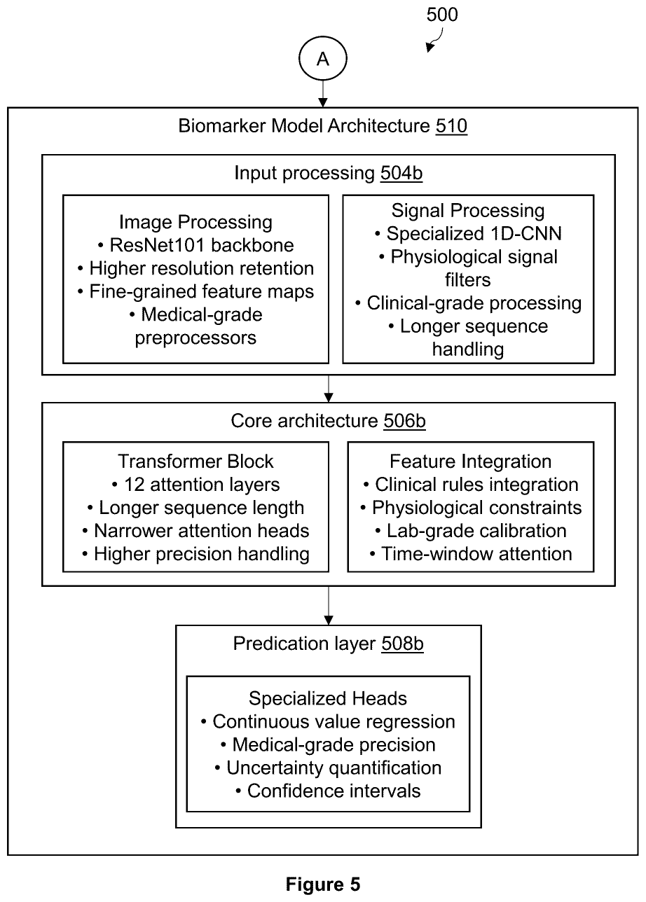

FIG. 5 illustrates an exemplary machine learning specific flow architecture 500, in accordance with an embodiment of the present disclosure.

In an exemplary embodiment, the machine learning specific flow architecture 500 includes a mental marker architecture 502 and a biomarker model architecture 510. The mental marker architecture 502 and the biomarker model architecture 510 are built on the data preprocessing subsystem 208. The mental marker architecture 502 comprises a first input processing layer 504a, a first core architecture 506a, and a first prediction layer 508a.

The first input processing layer 504a includes the colored image data processing and the speech signal processing, focusing on real-time and behavior-centric optimizations. The colored image data processing utilizes an EfficientNet backbone for motion-optimized real-time processing with an emphasis on temporal features. This ensures the extraction of actionable visual patterns relevant to the mental markers, such as facial expressions and movement cues. The speech signal processing employs a parallel 1D-CNN alongside one or more real-time filters to focus on behavioral patterns and handle shorter sequences. This is optimized for signals such as EEG and other neurophysiological data, ensuring rapid and targeted analysis.

The first core architecture 506a integrates processed inputs through a transformer block and feature integration. The transformer block comprises six attention layers configured for shorter sequences with broader attention heads. The transformer block is configured to focus on pattern recognition, which is crucial for detecting subtle mental and behavioral trends. The feature integration merges behavioral patterns over time while emphasizing temporal correlation. The feature integration includes cross-attention fusion that enables combining diverse data sources (the colored image data and the speech signal) to derive the one or more physiological indicators.

The first prediction layer 508a focuses on actionable outputs through multi-task capabilities. The first prediction layer 508a includes multi-task heads that perform classification and regression tasks, ensuring flexibility in the types of predictions (e.g., mental state classification, emotional analysis). The first prediction layer 508a includes at least one of: pattern stability checks for monitoring consistent behavioral trends, real-time confidence assessments to evaluate prediction reliability on the fly, state transition modeling to track changes in mental states over time, and the like.

The biomarker model architecture 510 comprises a second input processing layer 504b, a second core architecture 506b, and a second prediction layer 508b. The second input processing layer 504b includes the colored image data processing and the speech signal processing. The colored image data processing utilizes a ResNet101 backbone for extracting fine-grained feature maps while retaining high resolution. The colored image data processing employs a medical-grade preprocessor that ensures medical-grade accuracy by handling intricate visual details crucial for analysis. The signal processing employs a specialized 1D-CNN and physiological signal filters for processing longer temporal sequences. This enables accurate analysis of clinical signals, ensuring clinical-grade precision.

The second core architecture 506b includes the transformer block and the feature integration. The transformer block includes twelve attention layers, capable of processing longer sequences with narrow attention heads for higher precision. This allows detailed analysis of complex temporal patterns. The feature integration integrates features with clinical rules, physiological constraints, and lab-grade calibration. The feature integration also includes time-window attention that ensures the one or more ML models focus on critical temporal segments for accurate aggregation of the one or more multi-modal features.

The second prediction layer 508b employs specialized heads configured for continuous value regression, ensuring medical-grade precision. The second prediction layer 508b incorporates at least one of: uncertainty quantification to assess prediction reliability, confidence intervals for robust interpretability, and the like, critical for medical decision-making.

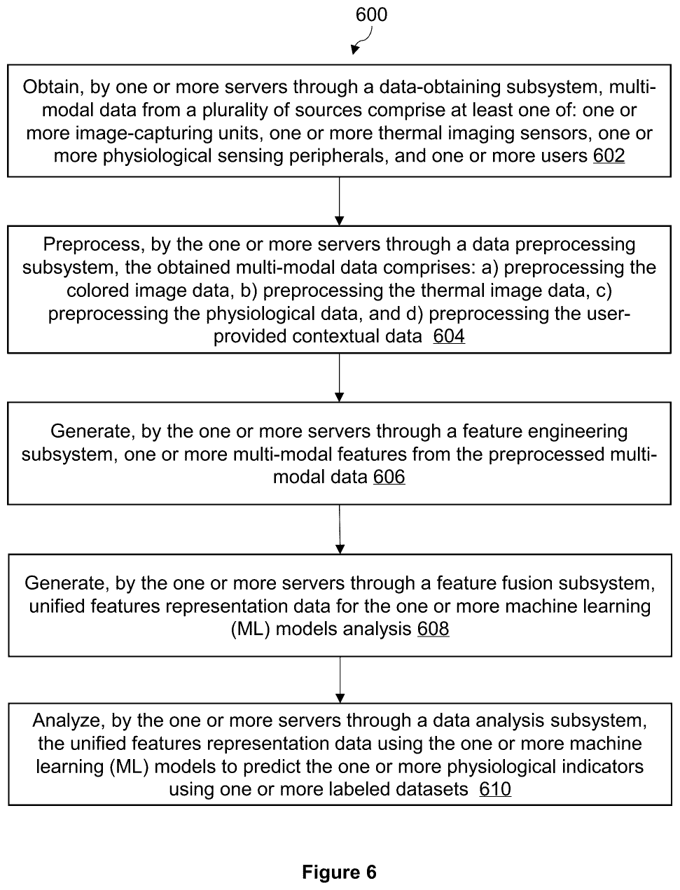

FIG. 6 illustrates an exemplary flow chart of a cognitive computing-based method 600 for non-invasive analysis of the one or more physiological indicators using the assisted transdermal optical imaging, in accordance with an embodiment of the present disclosure.

According to an exemplary embodiment of the present disclosure, the cognitive computing-based method 600 for non-invasive analysis of the one or more physiological indicators using the assisted transdermal optical imaging is disclosed. At step 602, the cognitive computing-based method 600 includes obtaining the multi-modal data from the plurality of sources by the one or more servers through the data-obtaining subsystem. This comprehensive and synchronized multi-modal data collection ensures a robust foundation for non-invasive analysis of the one or more physiological indicators.

The plurality of sources may comprise, but not constrained to, at least one of: the one or more image-capturing units, the one or more thermal imaging sensors, the one or more physiological sensing peripherals, the one or more users, and the like. The multi-modal data may comprise, but not restricted to, at least one of: the colored image data, the thermal image data, the physiological data, the speech signals, the user-provided contextual data, and the like. The one or more image-capturing units may comprise, but not limited to, at least one of: the high-resolution optical camera, the NIR camera, the hyperspectral imaging sensor, the multispectral camera, the ToF camera, the PPG imaging sensor, the polarized light camera, the micro-imaging sensor, and the like. The one or more thermal imaging sensors may comprise, but not constrained to, at least one of: the infrared thermal imaging sensor, the LWIR sensor, the MWIR sensor, the SWIR sensor, the FIR sensor, the microbolometer-based thermal sensor, the thermopile array sensor, the thermal imaging sensor integrated with visible-light imaging, and the like.

The one or more physiological sensing peripherals may comprise, but not restricted to, at least one of: the heart rate monitors, the blood pressure monitors, the respiratory rate sensors, the pulse oximeters, the body weighing scales, the body impedance analyzers, and the like. The colored image data may comprise, but not limited to, at least one of: the pixel-level blood flow patterns, the hemoglobin concentration, the oxygen saturation, the micro-expression detection data, and the like, in the defined facial regions, including at least one of: the cheeks, the forehead, nose, nasal cavity, the inner eyelids, and the like. The thermal image data may comprise, but not limited to, at least one of: the temperature distribution patterns, the inflammation indicators, the deeper tissue activity data, the blood flow dynamics, and the like, across the facial regions of the one or more users. The physiological data may comprise, but not limited to, at least one of: the heart rate data, the blood pressure data, the respiratory rate data, the oxygen level data, the body weight data, body temperature, the body impedance data, and the like. The user-provided contextual data may comprise, but not restricted to, at least one of: the user-reported stress levels; the sleep duration; the lifestyle factors, including at least one of: the physical activity level, the dietary habits, and the like; the medication history; the self-reported symptoms, including at least one of: the fatigue, the dizziness, the pain, and the like; the personal medical history, including at least one of: the pre-existing conditions, the diagnoses, and the like; the demographic information including at least one of: the age, the gender, the occupation, and the like.

At step 604, the cognitive computing-based method 600 includes preprocessing through the data preprocessing subsystem managed by the one or more servers. For the colored image data, the preprocessing includes performing at least one of: the face detection and alignment to standardize the positioning of the facial features, the motion compensation to account for movements during image capture, the artifact removal to eliminate noise and distortions, the standardization to normalize data, the ROI isolation to focus exclusively on the facial regions relevant for analysis, and the like. Preprocessing the colored image data comprises determining the facial regions of the one or more users using the one or more object detection models. The one or more object detection models are configured to ensure that the ROI is consistent across the one or more image frames in the colored image data.

Further, the colored image data is preprocessed by performing at least one of: the motion compensation process and the ROI tracking on the colored image data for stabilizing the ROI across the one or more image frames. The preprocessing of the colored image data further includes normalizing at least one of: the brightness, the contrast, the color, and the like of the colored image data associated to account for differences in the lighting conditions. Additionally, the colored image data is preprocessed by scaling the one or more images in the colored image data to the defined dimension for providing the uniform input for the one or more ML models.

For the thermal image data, the preprocessing includes the temperature calibration to adjust the sensor variations, the noise reduction to eliminate interference from environmental and sensor-based artifacts, the ROI tracking to pinpoint and follow specific areas over time.

The preprocessing of the thermal image data allows for precise capture of vital thermal indicators such as the temperature patterns, the blood flow, the potential inflammation markers, and the like. The preprocessing of the thermal image data further comprises aligning the thermal image data with the corresponding-colored image data by mapping the ROI to ensure the spatial and temporal consistency across the multi-modal data.

Similarly, the physiological data undergoes the preprocessing that includes the signal filtering to remove extraneous noise, the baseline correction to standardize signal levels, the feature extraction from raw signals, and the like. Additionally, the data is synchronized with at least of: the timestamps and the one or more physiological event markers, the outlier detection and exclusion, the unit standardization, the time normalization, the segmentation, and the like, of the physiological data into the predefined intervals to ensure uniformity and usability. The physiological data is preprocessed using at least one of: the Z-score normalization model, the min-max scaling model, the robust scaling model for at least one of: normalizing the physiological data, standardizing the physiological data, and performing the outlier detection and exclusion, and the like.

The user-provided contextual data is preprocessed through at least one of: the data encoding, authorizing the user-provided contextual data to validate the input integrity, computing the composite scores, and the like. The preprocessing of the user-provided contextual data further comprises processing the user-provided contextual data by using at least one of: the one-hot encoding model, the ordinal encoding model, the binary encoding model, and the like, as the one or more ML models for at least one of: converting into the categorical responses, converting into the scaled responses, processing one of: the yes and no contextual data, and the like. The preprocessing of the user-provided contextual data further comprises extracting at least one of: the symptom severity scores, the risk factor aggregation, the temporal change in the user-provided contextual data, the combined health indicators, the mental state indicators of the one or more users, and the like based on the processed user-provided contextual data. Preprocessing workflows collectively enhance the reliability and integration of the multi-modal data, setting a stage for non-invasive analysis of the one or more physiological indicators.

At step 606, the cognitive computing-based method 600 includes the feature engineering subsystem, facilitated by the one or more servers, to generate the one or more multi-modal features. The generation of the one or more multi-modal features begins with the extraction of at least one of: the blood flow pattern data, the hemoglobin estimation data, the tissue perfusion characteristics, the micro-expression detection data, and the like, from the preprocessed colored image data. The extraction is based on analyzing at least one of: the pixel-level changes, the color spectrum, the subtle facial movements, and the like.

To generate the one or more multi-modal features, the cognitive computing-based method 600 includes processing the thermal image data to extract at least one of: the temperature distribution patterns, the inflammation indicators, the blood flow dynamics, and the like, using at least one of: the one or more ML models, the image filtering models, and the like.

At least one of: the one or more ML models, the image filtering models, and the like, accurately capture and quantify thermal characteristics indicative of physiological and pathological changes.

Furthermore, from the physiological data, the cognitive computing-based method 600 generates at least one of: the time-domain HRV features, the frequency-domain HRV features, the systolic and diastolic blood pressure ratios, the respiratory variability, the stress and cardiovascular markers, the oxygen saturation data, and the like. The cognitive computing-based method 600 includes generating at least one of: the time-domain HRV features and the frequency-domain HRV features, including the first-frequency, the second-frequency, and the first-frequency and the second-frequency ratios, to evaluate the autonomic nervous system activity and stress and the cardiovascular markers. These features are extracted using the one or more ML models specifically trained to analyze physiological signals. Moreover, the cognitive computing-based method 600 processes the user-provided contextual data to determine at least one of: the encoded features, the derived composite metrics, and the like. Based on the features extracted from the colored image data and the thermal image data, generated features from the physiological data, and determined features from the user-provided contextual data, the one or more multi-modal features are generated. The cognitive computing-based method 600 includes generating the one or more multi-modal features using the one or more CNNs as the one or more ML models.

At step 608, the cognitive computing-based method 600 includes generating, through the feature fusion subsystem supported by the one or more servers, the unified features representation data. For generating the unified features representation data, the cognitive computing-based method 600 includes synchronizing the multi-modal data obtained from the plurality of sources. The synchronization is achieved by leveraging at least one of: the timestamps, the one or more physiological event markers, and the like, as the one or more anchor points. The one or more anchor points ensure that data streams with differing sampling rates and time alignments are harmonized, creating a temporally coherent dataset for further processing.

Following synchronization, the cognitive computing-based method 600 includes integrating the generated one or more multi-modal features into the unified representation features representation data. This integration enables the one or more ML models to simultaneously consider interrelations and correlations across modalities, enriching the analytical capabilities of the system. To enhance the one or more physiologically inapt combinations, the cognitive computing-based method 600 includes assigning the one or more domain-specific constraints to the unified features representation data.

The cognitive computing-based method 600 includes generating the unified features representation data by using at least one of: the one or more modality-specific attention procedures, the cross-modal relationship learning model, the one or more adaptive feature importance weighting procedures, and the like to: a) integrate the generated one or more multi-modal features into the unified features representation data, and b) assign the one or more weights to each multi-modal feature of the one or more multi-modal features based on reliability and relevance of the one or more multi-modal features for predicting the one or more physiological indicators.

At step 610, the cognitive computing-based method 600 includes analyzing the unified features representation data through the data analysis subsystem powered by the one or more servers and the one or more ML models. This analysis begins by performing the non-invasive analysis on the unified features representation data. This step 610 involves generating the data correlation between the unified features representation data and the one or more labeled datasets, enabling the prediction of the one or more physiological indicators.

The analysis further extends to detecting the one or more temporal trends within the unified features representation data. The one or more temporal trends encompass at least one of: change in the one or more physiological indicators over time, identification of the recurring patterns, the detection of the one or more abnormal health conditions (arrhythmias, inflammation, and early warning signs of chronic illnesses), and the like.

Moreover, the unified features representation data is analyzed by generating at least one of: the one or more numerical predictive insights (e.g., heart rate and oxygen saturation levels), the visual representations data (e.g., trend graphs and heatmaps), the one or more categorical predictive insights (e.g., classification of health conditions), the recommendation data (e.g., lifestyle modifications and medical consultations), and the like.

The one or more physiological indicators may comprise, but not constrained to, at least one of: the hemoglobin concentration, the HbA1c, the blood pressure, the blood glucose, the HRV, the SpO2, the respiratory rate, the stress levels, the cognitive load, the fatigue, the emotional variability, and the like. The one or more ML models may comprise, but not restricted to, at least one of: the XGBoost Regressor, the one or more CNNs with the LSTM hybrid, the random forest, the BiLSTM, the transformer-CNN, the RNNs, the LightGBM, the transformer-based multi-task model, the deep neural network, the gradient boosting, the neural networks, the k-means clustering, the hierarchical clustering, the support vector machines, and the like.

The data analysis subsystem is configured to select the one or more ML models based on the type of physiological indicators being predicted within the one or more physiological indicators. The one or more machine learning ML models may include, but not restricted to, at least one of: the one or more supervised learning models for the labeled data within the multi-modal data, the one or more unsupervised learning models for the unlabeled data within the multi-modal data, the one or more deep learning models, and the like.

The one or more supervised learning models may comprise, but not limited to, at least one of: the one or more regression models for determining at least one of: the hemoglobin concentration and the HbA1c, using at least one of: the XGBoost Regressor and the random forest, and the one or more classification models for determining the categorical outputs using at least one of: the SVMs and the one or more CNNs. The categorical outputs may comprise, but not constrained to, at least one of: the stress levels, the inflammation markers, and the like.

The one or more unsupervised learning models may include, but not restricted to, at least one of: the one or more clustering models that comprise at least one of: the k-means clustering and the hierarchical clustering, for identifying the one or more multi-modal feature patterns within the unified features representation data of the one or more users based on the one or more multi-modal features extracted from the multi-modal data.

The one or more deep learning models may include, but not restricted to, at least one of: the RNNs, the transformer-CNN, and the LSTM hybrid, for analyzing at least one of: the blood flow patterns, the hemoglobin estimation data, the tissue perfusion characteristics, the micro-expression detection data, and the like, in the colored image data.

Moreover, the cognitive computing-based method 600 includes training the one or more ML models to ensure accurate and reliable analysis of the multi-modal data. This training involves the use of at least one of: the one or more loss function models, the SGD, the Adam, the RMSprop, the Hyperparameter tuning models, and the like. These techniques are employed to achieve precise outcomes in various tasks, including at least one of: the prediction of the continuous variables, classification of the multi-modal data, the mitigation of the one or more physiologically inapt combinations, and the like.

The cognitive computing-based method 600 includes adaptive retraining of the one or more ML models on the real-time acquired multi-modal data. This retraining process addresses dynamic changes, such as the multi-modal data drift caused by the variations in at least one of: the demographic information, the environmental conditions, the one or more sensor settings, and the like. By continuously learning from the real-time acquired multi-modal data, the one or more ML models are optimized to maintain optimal prediction.

Furthermore, the cognitive computing-based method 600 incorporates the one or more feedback loops from the one or more users, thereby allowing refinement of the predictions and the outputs of the one or more ML models over time.

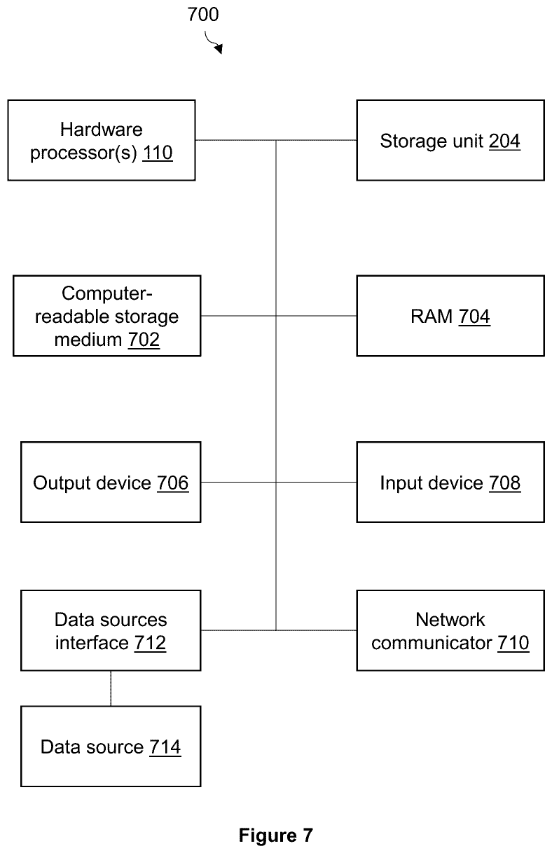

FIG. 7 illustrates an exemplary block diagram representation of one or more server platforms 700 for implementation of the disclosed system 102, in accordance with an embodiment of the present disclosure.

In an exemplary embodiment, for the sake of brevity, the construction, and operational features of the system 102 which are explained in detail above are not explained in detail herein. Particularly, computing machines such as but not limited to internal/external server clusters, quantum computers, desktops, laptops, smartphones, tablets, and wearables may be used to execute the system 102 or may include the structure of the one or more server platforms 700. As illustrated, the one or more server platforms 700 may include additional components not shown, and some of the components described may be removed and/or modified. For example, a computer system with the multiple graphics processing units (GPUs) may be located on at least one of: internal printed circuit boards (PCBs) and external-cloud platforms including Amazon® Web Services (AWS), Google® Cloud Platform (GCP) Microsoft® Azure (Azure), internal corporate cloud computing clusters, or organizational computing resources.

The one or more server platforms 700 may be a computer system such as the system 102 that may be used with the embodiments described herein. The computer system may represent a computational platform that includes components that may be in the one or more servers 124 or another computer system. The computer system may be executed by the one or more hardware processors 110 (e.g., single, or multiple processors) or other hardware processing circuits, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the one or more hardware processors 110 that execute software instructions or code stored on a non-transitory computer-readable storage medium 702 to perform methods of the present disclosure. The software code includes, for example, instructions to gather data and analyze the multi-modal data. For example, the plurality of subsystems 114 includes the data-obtaining subsystem 206, the data preprocessing subsystem 208, the feature engineering subsystem 210, the feature fusion subsystem 212, and the data analysis subsystem 214.

The instructions on the computer-readable storage medium 702 are read and stored the instructions in the storage unit or random-access memory (RAM) 704. The storage unit 204 may provide a space for keeping static data where at least some instructions could be stored for later execution. The stored instructions may be further compiled to generate other representations of the instructions and dynamically stored in the RAM 704. The one or more hardware processors 110 may read instructions from the RAM 704 and perform actions as instructed.

The computer system may further include an output device 706 to provide at least some of the results of the execution as output including, but not limited to, visual information of the one or more physiological indicators to the one or more users. The output device 706 may include a display on computing devices and virtual reality glasses. For example, the display may be a mobile phone screen or a laptop screen. Graphical user interfaces (GUIs) and/or text may be presented as an output on the display screen. The computer system may further include an input device 708 to provide the one or more users or another device with mechanisms for entering the user-provided contextual data and/or otherwise interacting with the computer system. The input device 708 may include, for example, a keyboard, a keypad, a mouse, or a touchscreen. Each of the output devices 706 and the input device 708 may be joined by one or more additional peripherals.

A network communicator 710 may be provided to connect the computer system to a network and in turn to other devices connected to the network including other entities, servers, data stores, and interfaces. The network communicator 710 may include, for example, a network adapter such as a LAN adapter or a wireless adapter. The computer system may include a data sources interface 712 to access a data source 714. The data source 714 may be an information resource about the one or more ML models. As an example, the one or more databases 104 of exceptions and rules may be provided as the data source 714. Moreover, knowledge repositories and curated data may be other examples of the data source 714. The data source 714 may include libraries containing, but not limited to, datasets related to the one or more ML models, the ML model configurations, historical data, and other essential information. Moreover, the data sources interface 712 enables the system 102 to dynamically access and update these data repositories as new information is collected, analyzed, and utilized.

Numerous advantages of the present disclosure may be apparent from the discussion above. In accordance with the present disclosure, the system for non-invasive analysis of the one or more physiological indicators using the assisted transdermal optical imaging. The system is implemented by including multiple ways to collect the multi-modal data in real time and fusing the collected multi-modal data to achieve higher accuracy. Additionally, an increase in the quantity of multi-modal data, results in lesser dependency on the optical image, thereby resulting in lesser influence of operating conditions. The system enables measurements at home, measurements at clinics, hospitals, laboratories, and the like. The system enables the one or more users anywhere and anytime health monitoring. The system enables the one or more users to determine the one or more physiological indicators at home with the one or more image-capturing units. The one or more image-capturing units may be a mobile phone camera. Additionally, easily available vital measurement devices such as—the BP monitor, the pulse oximeter, the thermometer, the body composition scale, and the microphone make the system user-friendly and easily available.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices that are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limited, of the scope of the invention, which is set forth in the following claims.

What is claimed is:

1. A cognitive computing-based method for non-invasive analysis of one or more physiological indicators using assisted transdermal optical imaging, comprising:
obtaining, by one or more servers through a data-obtaining subsystem, multi-modal data from a plurality of sources comprise at least one of: one or more image-capturing units, one or more thermal imaging sensors, one or more physiological sensing peripherals, and one or more users,
wherein the multi-modal data comprises at least one of: colored image data, thermal image data, physiological data, speech signals, and user-provided contextual data;
preprocessing, by the one or more servers through a data preprocessing subsystem, the obtained multi-modal data comprises:
preprocessing the colored image data based on performing at least one of: a face detection and alignment, a motion compensation, a standardization, an artifact removal, and a region-of-interest (ROI) isolation;
preprocessing the thermal image data based on performing at least one of: a temperature calibration, a noise reduction, and a region-of-interest (ROI) tracking to capture at least one of: temperature patterns, blood flow, and inflammation markers;
preprocessing the physiological data based on performing at least one of: a signal filtering, a baseline correction, a feature extraction for raw signals, a data synchronization with at least one of: timestamps and one or more physiological event markers, outlier detection and exclusion, time normalization, unit standardization, and segmentation of the physiological data at pre-defined intervals; and
preprocessing the user-provided contextual data to perform at least one of: data encoding, authorizing contextual data, and computing composite scores;
generating, by the one or more servers through a feature engineering subsystem, one or more multi-modal features based on:
extracting at least one of: blood flow pattern data, hemoglobin estimation data, tissue perfusion characteristics, and micro-expression detection data from the colored image data based on analyzing at least one of: pixel-level changes, a color spectrum, and subtle facial movements, in the preprocessed colored image data;
extracting at least one of: temperature distribution patterns, inflammation indicator data, and blood flow dynamics from the thermal image data using at least one of: one or more machine learning (ML) models and one or more image filtering models;
generating at least one of: time-domain heart rate variability (HRV) features, frequency-domain heart rate variability (HRV) features, systolic and diastolic blood pressure ratios, respiratory variability, stress and cardiovascular markers, and oxygen saturation fluctuation data from the physiological data using the one or more machine learning (ML) models; and
determining at least one of: encoded features and derived metrics from the user-provided contextual data;
generating, by the one or more servers through a feature fusion subsystem, unified features representation data comprises:
synchronizing the multi-modal data from the plurality of sources using at least one of: the timestamps and the one or more physiological event markers as one or more anchor points;
integrating the generated one or more multi-modal features into the unified features representation data for the one or more machine learning (ML) models analysis; and
assigning one or more domain-specific constraints to the unified features representation data to alleviate one or more physiologically inapt combinations; and
analyzing, by the one or more servers through a data analysis subsystem, the unified features representation data using the one or more machine learning (ML) models comprises:
performing non-invasive analysis on the unified features representation data based on generating a data correlation with one or more labeled datasets to predict the one or more physiological indicators,
the one or more physiological indicators comprises at least one of: hemoglobin concentration, glycated hemoglobin (HbA1c), blood pressure, blood glucose, heart rate variability (HRV), blood oxygen saturation (SpO₂), respiratory rate, stress levels, cognitive load, fatigue, and emotional variability;
detecting one or more temporal trends in the unified features representation data including at least one of: change in the one or more physiological indicators over time, recurring patterns in the physiological data, and one or more abnormal health conditions; and
generating at least one of: one or more numerical predictive insights, visual representations data, one or more categorical predictive insights, and recommendation data of the one or more physiological indicators based on the non-invasive analysis of the one or more physiological indicators.

2. The cognitive computing-based method of claim 1, wherein
the one or more image-capturing units comprise at least one of: a high-resolution optical camera, a near-infrared (NIR) camera, a hyperspectral imaging sensor, a multispectral camera, a time-of-flight (ToF) camera, a photoplethysmography (PPG) imaging sensor, a polarized light camera, and a micro-imaging sensor;
the one or more thermal imaging sensors comprise at least one of: an infrared thermal imaging sensor, a long-wave infrared (LWIR) sensor, a mid-wave infrared (MWIR) sensor, a short-wave infrared (SWIR) sensor, a far-infrared (FIR) sensor, a microbolometer-based thermal sensor, a thermopile array sensor, and a thermal imaging sensor integrated with visible-light imaging; and
the one or more physiological sensing peripherals comprises at least one of: heart rate monitors, blood pressure monitors, electrocardiogram (ECG), Non-Contact IR Thermometer (NCIT), respiratory rate sensors, pulse oximeters, body weighing scales, and body impedance analyzers.

3. The cognitive computing-based method of claim 1, wherein
the colored image data comprises at least one of: pixel-level blood flow patterns, hemoglobin concentration, oxygen saturation, and the micro-expression detection data in defined facial regions, including at least one of: cheeks, a forehead, nose, nasal cavity, and inner eyelids;
the thermal image data comprises at least one of: temperature distribution patterns, inflammation indicators, deeper tissue activity data, and blood flow dynamics across facial regions of the one or more users;
the physiological data comprises at least one of: heart rate data, blood pressure data, respiratory rate data, oxygen level data, body weight data, body temperature, and body impedance data;
the user-provided contextual data comprises at least one of: user-reported stress levels; sleep duration; lifestyle factors, including at least one of: physical activity level and dietary habits; medication history; self-reported symptoms, including at least one of: fatigue, dizziness, and pain; personal medical history, including at least one of: pre-existing conditions and diagnoses; and demographic information including at least one of: age, gender, and occupation.

4. The cognitive computing-based method of claim 1, wherein obtaining the speech signals using one or more speech processors to extract one or more acoustic features indicative of at least one of: emotional variability, fatigue, and stress levels,
the one or more acoustic features comprise at least one of: a pitch, a tone, a speech rhythm, and a voice intensity, processed by the one or more machine learning (ML) models to determine the one or more physiological indicators.

5. The cognitive computing-based method of claim 1, wherein preprocessing the colored image data comprise at least one of:
determining the facial regions of the one or more users using one or more object detection models to ensure the region-of-interest (ROI) is consistent across one or more image frames in the colored image data;
performing at least one of: the motion compensation process and the region-of-interest (ROI) tracking on the colored image data for stabilizing the region-of-interest (ROI) across the one or more image frames;
normalizing at least one of: brightness, contrast, and color of the colored image data associated to account for differences in lighting conditions; and
scaling one or more images in the colored image data to a defined dimension for providing uniform input for the one or more machine learning (ML) models.

6. The cognitive computing-based method of claim 1, wherein preprocessing the thermal image data further comprises:
aligning the thermal image data with the corresponding-colored image data by mapping the regions of interest (ROI) to ensure spatial and temporal consistency across the multi-modal data.

7. The cognitive computing-based method of claim 1, wherein preprocessing the physiological data comprises:
processing the physiological data using at least one of: a Z-score normalization model, a min-max scaling model, a robust scaling model for at least one of: normalizing the physiological data, standardizing the physiological data, and perform the outlier detection and exclusion.

8. The cognitive computing-based method of claim 1, wherein preprocessing the user-provided contextual data further comprises:
processing the user-provided contextual data using at least one of: a one-hot encoding model, an ordinal encoding model, and a binary encoding model, as the one or more machine learning (ML) models for at least one of: converting into categorical responses, converting into scaled responses, and processing one of: yes and no contextual data; and
extracting at least one of: symptom severity scores, risk factor aggregation, temporal change in the contextual data, combined health indicators, and mental state indicators of the one or more users based on the processed user-provided contextual data.

9. The cognitive computing-based method of claim 1, wherein generating the one or more multi-modal features using one or more convolutional neural networks (CNNs) as the one or more machine learning (ML) models,
generating at least one of: the time-domain heart rate variability (HRV) features and the frequency-domain heart rate variability (HRV) features, including a first-frequency, a second-frequency, and the first-frequency and the second-frequency ratios, to evaluate autonomic nervous system activity and stress and cardiovascular markers.

10. The cognitive computing-based method of claim 1, wherein generating the unified features representation data comprises:
using at least one of: one or more modality-specific attention procedures, a cross-modal relationship learning model, one or more adaptive feature importance weighting procedures, to:
integrating the generated one or more multi-modal features into unified features representation data; and
assigning one or more weights to each multi-modal feature of the one or more multi-modal features based on reliability and relevance of the one or more multi-modal features for predicting the one or more physiological indicators.

11. The cognitive computing-based method of claim 1, wherein the one or more machine learning (ML) models comprises at least one of: an XGBoost Regressor, the one or more convolutional neural networks (CNNs) with a long short-term memory (LSTM) hybrid, a random forest, a bidirectional long short-term memory (BiLSTM), a transformer-convolutional neural network (CNN), recurrent neural networks (RNNs), light gradient-boosting machine (LightGBM), a transformer-based multi-task model, deep neural network, gradient boosting, neural networks, a k-means clustering, a hierarchical clustering and support vector machines (SVMs).

12. The cognitive computing-based method of claim 1, wherein the data analysis subsystem is configured to select the one or more machine learning (ML) models based on a type of physiological indicators being predicted within the one or more physiological indicators, wherein the one or more machine learning (ML) models include at least one of:
one or more supervised learning models for labeled data within the multi-modal data, including:
one or more regression models for determining at least one of: the hemoglobin concentration and the glycated hemoglobin (HbA1c), using at least one of: the XGBoost Regressor and the random forest; and
one or more classification models for determining categorical outputs, comprise at least one of: the stress levels and inflammation markers, using at least one of: the support vector machines (SVMs) and the one or more convolutional neural networks (CNNs);
one or more unsupervised learning models for unlabeled data within the multi-modal data, including:
one or more clustering models comprises at least one of: the k-means clustering and the hierarchical clustering, for identifying one or more multi-modal feature patterns within the unified features representation data of the one or more users based on the one or more multi-modal features extracted from the multi-modal data; and
one or more deep learning models including at least one of: the recurrent neural networks (RNNs), the transformer-convolutional neural network (CNN), and the long short-term memory (LSTM) hybrid, for analyzing at least one of: the blood flow patterns, the hemoglobin estimation data, the tissue perfusion characteristics, and the micro-expression detection data in the colored image data.

13. The cognitive computing-based method of claim 1, further comprising:
training the one or more machine learning (ML) models using at least one of: one or more loss function models, stochastic gradient descent (SGD), Adaptive Moment Estimation (Adam), Root Mean Square Propagation (RMSprop), Hyperparameter tuning models, for at least one of: continuous variable prediction, classification of the multi-modal data, and alleviate the one or more physiologically inapt combinations;
adaptively retraining the one or more machine learning (ML) models on real-time acquired multi-modal data to optimize prediction accuracy and address multi-modal data drift caused by variations in at least one of: the demographic information, environmental conditions, and one or more sensor settings; and
incorporating one or more feedback loops from the one or more users to refine the predictions and outputs of the one or more machine learning (ML) models over time.

14. A cognitive computing-based system for non-invasive analysis of one or more physiological indicators using assisted transdermal optical imaging, comprising:
one or more servers, comprising:
one or more hardware processors; and
a memory unit operatively connected to the one or more hardware processors, wherein the memory unit comprises a set of computer-readable instructions in form of a plurality of subsystems, configured to be executed by the one or more hardware processors, wherein the plurality of subsystems comprises:
a data-obtaining subsystem configured to obtain multi-modal data from a plurality of sources comprise at least one of: one or more image-capturing units, one or more thermal imaging sensors, one or more physiological sensing peripherals, and one or more users;
wherein the multi-modal data comprises at least one of: colored image data, thermal image data, physiological data, speech signals, and user-provided contextual data.
a data preprocessing subsystem configured to:
preprocess the colored image data based on performing at least one of: a face detection and alignment, a motion compensation, a standardization, an artifact removal, and a region-of-interest (ROI) isolation;
preprocess the thermal image data based on performing at least one of: a temperature calibration, a noise reduction, a region-of-interest (ROI) tracking to capture at least one of: temperature patterns, blood flow, and inflammation markers;
preprocess the physiological data based on performing at least one of: a signal filtering, a baseline correction, a feature extraction for raw signals, a data synchronization with at least one of: timestamps and one or more physiological event markers, outlier detection and exclusion, time normalization, unit standardization, and segmentation of the physiological data at pre-defined intervals; and
preprocess the user-provided contextual data to perform at least one of: data encoding, authorizing contextual data, and computing composite scores;
a feature engineering subsystem configured to generate one or more multi-modal features based on:
extracting at least one of: blood flow pattern data, hemoglobin estimation data, tissue perfusion characteristics, and micro-expression detection data from the colored image data based on analyzing at least one of: pixel-level changes, a color spectrum, and subtle facial movements, in the preprocessed colored image data;
extracting at least one of: temperature distribution patterns, inflammation indicator data, and blood flow dynamics from the thermal image data using at least one of: one or more machine learning (ML) models and one or more image filtering models;
generating at least one of: time-domain heart rate variability (HRV) features, frequency-domain heart rate variability (HRV) features, systolic and diastolic blood pressure ratios, respiratory variability, stress and cardiovascular markers, and oxygen saturation fluctuation data from the physiological data using the one or more machine learning (ML) models; and
determining at least one of: encoded features and derived metrics from the user-provided contextual data;
a feature fusion subsystem configured to:
synchronize the multi-modal data from the plurality of sources using at least one of: the timestamps and the one or more physiological event markers as one or more anchor points;
integrate the generated one or more multi-modal features into unified features representation data for the one or more machine learning (ML) models analysis; and
assigning one or more domain-specific constraints to the unified features representation data to alleviate one or more physiologically inapt combinations; and
a data analysis subsystem configured with the one or more machine learning (ML) models to:
perform non-invasive analysis on the unified features representation data based on generating a data correlation with one or more labeled datasets to predict the one or more physiological indicators,
the one or more physiological indicators comprises at least one of: hemoglobin concentration, glycated hemoglobin (HbA1c), blood pressure, blood glucose, heart rate variability (HRV), blood oxygen saturation (SpO₂), respiratory rate, stress levels, cognitive load, fatigue, and emotional variability;
detect one or more temporal trends in the unified features representation data including at least one of: change in the one or more physiological indicators over time, recurring patterns in the physiological data, and one or more abnormal health conditions; and
generate at least one of: one or more numerical predictive insights, visual representations data, one or more categorical predictive insights, and recommendation data of the one or more physiological indicators based on the non-invasive analysis of the one or more physiological indicators.

15. The cognitive computing-based system of claim 14, wherein
the colored image data comprises at least one of: pixel-level blood flow patterns, hemoglobin concentration, oxygen saturation, and the micro-expression detection data in defined facial regions, including at least one of: cheeks, a forehead, nose, nasal cavity, and inner eyelids;
the thermal image data comprises at least one of: temperature distribution patterns, inflammation indicators, and blood flow dynamics across facial regions of the one or more users;
the physiological data comprises at least one of: heart rate data, blood pressure data, respiratory rate data, oxygen level data, body weight data, and body impedance data;
the user-provided contextual data comprises at least one of: user-reported stress levels; sleep duration; lifestyle factors, including at least one of: physical activity level and dietary habits; medication history; self-reported symptoms, including at least one of: fatigue, dizziness, and pain; personal medical history, including at least one of: pre-existing conditions and diagnoses; and demographic information including at least one of: age, gender, and occupation.

16. The cognitive computing-based system of claim 14, wherein the colored image data preprocessing comprise at least one of:
determine the facial regions of the one or more users using one or more object detection models to ensure the region-of-interest (ROI) is consistent across one or more image frames in the colored image data;
perform at least one of: the motion compensation process and the region-of-interest (ROI) tracking on the colored image data for stabilizing the region-of-interest (ROI) across the one or more image frames;
normalize at least one of: brightness, contrast, and color of the colored image data associated to account for differences in lighting conditions; and
scale one or more images in the colored image data to a defined dimension for providing uniform input for the one or more machine learning (ML) models.

17. The cognitive computing-based system of claim 14, wherein the thermal image data preprocessing comprises:
align the thermal image data with the corresponding-colored image data by mapping the regions of interest (ROI) to ensure spatial and temporal consistency across the multi-modal data.

18. The cognitive computing-based system of claim 14, wherein the data analysis subsystem is configured to select the one or more machine learning (ML) models based on a type of physiological indicators being predicted within the one or more physiological indicators, wherein the one or more machine learning (ML) models include at least one of:
one or more supervised learning models for labeled data within the multi-modal data, including:
one or more regression models for determining at least one of: the hemoglobin concentration and the glycated hemoglobin (HbA1c), using at least one of: the XGBoost Regressor and the random forest; and
one or more classification models for determining categorical outputs, comprise at least one of: the stress levels and inflammation markers, using at least one of: the support vector machines (SVMs) and the one or more convolutional neural networks (CNNs);
one or more unsupervised learning models for unlabeled data within the multi-modal data, including:
one or more clustering models comprises at least one of: the k-means clustering and the hierarchical clustering, for identifying one or more multi-modal feature patterns within the unified features representation data of the one or more users based on the one or more multi-modal features extracted from the multi-modal data.
one or more deep learning models including at least one of: the recurrent neural networks (RNNs), the transformer-convolutional neural network (CNN), and the long short-term memory (LSTM) hybrid, for analyzing at least one of: the blood flow patterns, the hemoglobin estimation data, the tissue perfusion characteristics, and the micro-expression detection data in the colored image data.

19. The cognitive computing-based system of claim 14, wherein the one or more machine learning (ML) models are configured to:
train using at least one of: one or more loss function models, stochastic gradient descent (SGD), Adaptive Moment Estimation (Adam), Root Mean Square Propagation (RMSprop), Hyperparameter tuning models, for at least one of: continuous variable prediction, classification of the multi-modal data, and alleviate the one or more physiologically inapt combinations;
adaptively retrain on real-time acquired multi-modal data to optimize prediction accuracy and address multi-modal data drift caused by variations in at least one of: the demographic information, environmental conditions, and one or more sensor settings; and
incorporate one or more feedback loops from the one or more users to refine the predictions and outputs of the one or more machine learning (ML) models over time.

20. A non-transitory computer-readable storage medium storing computer-executable instructions that, when executed by one or more hardware processors, cause the one or more hardware processors to perform operations for non-invasive analysis of one or more physiological indicators using assisted transdermal optical imaging, the operations comprising:
obtaining multi-modal data from a plurality of sources comprise at least one of: one or more image-capturing units, one or more thermal imaging sensors, one or more physiological sensing peripherals, and one or more users,
wherein the multi-modal data comprises at least one of: colored image data, thermal image data, physiological data, speech signals, and user-provided contextual data;
preprocessing the obtained multi-modal data comprises:
preprocessing the colored image data based on performing at least one of: a face detection and alignment, a motion compensation, a standardization, an artifact removal, and a region-of-interest (ROI) isolation;
preprocessing the thermal image data based on performing at least one of: a temperature calibration, a noise reduction, and a region-of-interest (ROI) tracking to capture at least one of: temperature patterns, blood flow, and inflammation markers;
preprocessing the physiological data based on performing at least one of: a signal filtering, a baseline correction, a feature extraction for raw signals, a data synchronization with at least one of: timestamps and one or more physiological event markers, outlier detection and exclusion, time normalization, unit standardization, and segmentation of the physiological data at pre-defined intervals; and
preprocessing the user-provided contextual data to perform at least one of: data encoding, authorizing contextual data, and computing composite scores;
generating one or more multi-modal features based on:
extracting at least one of: blood flow pattern data, hemoglobin estimation data, tissue perfusion characteristics, and micro-expression detection data from the colored image data based on analyzing at least one of: pixel-level changes, a color spectrum, and subtle facial movements, in the preprocessed colored image data;
extracting at least one of: temperature distribution patterns, inflammation indicator data, and blood flow dynamics from the thermal image data using at least one of: one or more machine learning (ML) models and image filtering models;
generating at least one of: time-domain heart rate variability (HRV) features, frequency-domain heart rate variability (HRV) features, systolic and diastolic blood pressure ratios, respiratory variability, stress and cardiovascular markers, and oxygen saturation fluctuation data from the physiological data using one or more machine learning (ML) models; and
determining at least one of: encoded features and derived metrics from the user-provided contextual data;
generating unified features representation data comprises:
synchronizing the multi-modal data from the plurality of sources using at least one of: the timestamps and the one or more physiological event markers as one or more anchor points;
integrating the generated one or more multi-modal features into the unified features representation data for the one or more machine learning (ML) models analysis; and
assigning one or more domain-specific constraints to the unified features representation data to alleviate one or more physiologically inapt combinations; and
analyzing the unified features representation data using the one or more machine learning (ML) models comprises:
performing non-invasive analysis on the unified features representation data based on generating a data correlation with one or more labeled datasets to predict the one or more physiological indicators,
the one or more physiological indicators comprise at least one of: hemoglobin concentration, glycated hemoglobin (HbA1c), blood pressure, blood glucose, heart rate variability (HRV), blood oxygen saturation (SpO₂), respiratory rate, stress levels, cognitive load, fatigue, and emotional variability;
detecting one or more temporal trends in the unified features representation data including at least one of: change in the one or more physiological indicators over time, recurring patterns in the physiological data, and one or more abnormal health conditions; and
generating at least one of: one or more numerical predictive insights, visual representations data, one or more categorical predictive insights, and recommendation data of the one or more physiological indicators based on the non-invasive analysis of the one or more physiological indicators.
